(19) United States
(12) Patent Application Publication
(10) Pub. No.: US 2006/0074046 A1
(43) Pub. Date: Apr. 6, 2006

(54) Oral administration of decitabine salt

(76) Inventors: Sanjeev Redkar, Hayward, CA (US); Pasit Phiasivongsa, Brentwood, CA (US)

Correspondence Address: WILSON SONSINI GOODRICH & ROSATI, 650 PAGE MILL ROAD, PALO ALTO, CA, 94304-1050, US

(21) Appl. No.: 11/238,168

(22) Filed: Sep. 27, 2005

Related U.S. Application Data
(63) Continuation-in-part of application No. 10/952,252, filed on Sep. 27, 2004.

(51) Int. Cl. A61K 31/7072; C07H 19/12

(52) U.S. Cl. 514/49; 536/28.3

(57) ABSTRACT

The present invention relates to salts of decitabine as well as methods for synthesizing the salts described herein. Pharmaceutical compositions and methods of using the decitabine salts are also provided, including methods of orally administering the salts or pharmaceutical compositions thereof to treat conditions, such as cancer and hematological disorders.

### CROSS-REFERENCE

This application is a continuation-in-part application of Ser. No. 10/952,252, filed Sep. 27, 2004, which is incorporated herein by reference in its entirety and to which application we claim priority under 35 USC § 120.

### STATEMENT AS TO FEDERALLY SPONSORED RESEARCH

This invention was made with the support of the United States government by the National Institutes of Health.

### BACKGROUND OF THE INVENTION

A few azacytosine nucleosides, such as 5-aza-2′-deoxycytidine (also called decitabine) and 5-azacytidine (also called azacitidine), have been developed as antagonist of its related natural nucleoside, 2′-deoxycytidine and cytidine, respectively. The only structural difference between azacytosine and cytosine is the presence of a nitrogen at position 5 of the cytosine ring in azacytosine as compared to a carbon at this position for cytosine.

Two isomeric forms of decitabine can be distinguished. The β-anomer is the active form. The modes of decomposition of decitabine in aqueous solution are (a) conversion of the active β-anomer to the inactive α-anomer (Pompon et al. (1987) J. Chromat. 388:113-122); (b) ring cleavage of the aza-pyrimidine ring to form N-(formylamidino)-N′-β-D-2′-deoxy-(ribofuranosy)-urea (Mojaverian and Repta (1984) J. Pharm. Pharmacol. 36:728-733); and (c) subsequent formation of guanidine compounds (Kissinger and Stemm (1986) J. Chromat. 353:309-318).

Decitabine possesses multiple pharmacological characteristics. At a molecular level, it is S-phase dependent for incorporation into DNA. At a cellular level, decitabine can induce cell differentiation and exert hematological toxicity. Despite having a short half-life in vivo, decitabine has an excellent tissue distribution.

One of the functions of decitabine is its ability to specifically and potently inhibit DNA methylation. Methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. Momparler et al. (1985) 30:287-299. After conversion to its triphosphate form by deoxycytidine kinase, decitabine is incorporated into replicating DNA at a rate similar to that of the natural substrate, dCTP. Bouchard and Momparler (1983) Mol. Pharmacol. 24:109-114.

Incorporation of decitabine into the DNA strand has a, hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. Juttermann et al. (1994) Proc. Natl. Acad. Sci. USA 91:11797-11801. By specifically inhibiting DNA methyltransferase, the enzyme required for methylation, the aberrant methylation of the tumor suppressor genes could be prevented.

Decitabine is commonly supplied as a sterile lyophilized powder for injection, together with buffering salt, such as potassium dihydrogen phosphate, and pH modifier, such as sodium hydroxide. For example, decitabine is supplied by SuperGen, Inc., as lyophilized powder packed in 20 mL glass vials, containing 50 mg of decitabine, monobasic potassium dihydrogen phosphate, and sodium hydroxide. When reconstituted with 10 mL of sterile water for injection, each mL contain 5 mg of decitabine, 6.8 mg of KH₂PO₄, and approximately 1.1 mg NaOH. The pH of the resulting solution is 6.5-7.5. The reconstituted solution can be further diluted to a concentration of 1.0 or 0.1 mg/mL in cold infusion i.e., 0.9% Sodium Chloride; or 5% Dextrose; or 5% Glucose; or Lactated Ringer's. The unopened vials are typically stored under refrigeration (2-8° C.; 36-46° F.), in the original package.

Decitabine is most typically administrated to patients by injection, such as by a bolus I.V. injection, continuous I.V. infusion, or I.V. infusion. Similar to decitabine, azacitidine is also formulated as aqueous solution and delivered to patients intravenously. According to clinical studies of azacitidine, longer or continuous infusions were more effective than shorter ones. Santini et al. (2001) Ann. Int. Med. 134: 573-588. However, the length of I.V. infusion is limited by the decomposition of decitabine or azacitidine and low solubility of the drugs in aqueous solutions. The present invention provides innovative solutions to such problems.

### SUMMARY OF THE INVENTION

According to the present invention, a salt of a cytidine analog is provided.

In one embodiment, the cytidine analog is 5-aza-2′-deoxycytidine or 5-azacytidine.

In another embodiment, the salt of the cytidine analog is synthesized with an acid, optionally with an acid having a pKₐ of about 5 or less, optionally with an acid having pKₐ of about 4 or less, optionally with an acid having pKₐ ranging from about 3 to about 0, or optionally with an acid having pKₐ ranging from about 3 to about −10.

Preferably, the acid is selected from the group consisting of hydrochloric, L-lactic, acetic, phosphoric, (+)-L-tartaric, citric, propionic, butyric, hexanoic, L-aspartic, L-glutamic, succinic, EDTA, maleic, methanesulfonic acid, HBr, HF, HI, nitric, nitrous, sulfuric, sulfurous, phosphorous, perchloric, chloric, chlorous acid, carboxylic acid, sulfonic acid, ascorbic, carbonic, and fumaric acid. In particular, the sulfonic acid is selected from the group consisting of ethanesulfonic, 2-hydroxyethanesulfonic, and toluenesulfonic acid.

In yet another embodiment, a salt of decitabine is provided. The salt of decitabine preferably is selected from the group consisting of hydrochloride, mesylate, EDTA, sulfite, L-Aspartate, maleate, phosphate, L-Glutamate, (+)-L-Tartrate, citrate, L-Lactate, succinate, acetate, hexanoate, butyrate, or propionate salt.

In one variation of the embodiment, the salt of decitabine is hydrochloride salt in crystalline form characterized by an X-ray diffraction pattern having diffraction peaks (2θ) at 14.79°, 23.63°, and 29.81°. The salt is further characterized by a melting endotherm of 125-155° C., optionally 130-144° C., as measured by differential scanning calorimetry at a scan rate of 10° C. per minute.

In another variation of the embodiment, the salt of decitabine is a mesylate salt in crystalline form characterized by an X-ray diffraction pattern having diffraction peaks (2θ) at 8.52°, 22.09°, and 25.93°. The salt is further characterized by a melting endotherm of 125-140° C., or optionally 125-134° C., as measured by differential scanning calorimetry at a scan rate of 10° C. per minute.

In yet another variation of the embodiment, the salt of decitabine is an EDTA salt in crystalline form characterized by an X-ray diffraction pattern having diffraction peaks (2θ) at 7.14°, 22.18°, and 24.63°. The salt is further characterized by multiple reversible melting endotherms at 50-90° C., 165-170° C., and 170-200° C., or optionally at 73° C., 169° C., and 197° C., as measured by differential scanning calorimetry at a scan rate of 10° C. per minute

In yet another variation of the embodiment, the salt of decitabine is a sulfite salt in crystalline form characterized by an X-ray diffraction pattern having diffraction peaks (2θ) at 15.73°, 19.23°, and 22.67°. The salt is further characterized by a melting endotherm at 100-140° C. as measured by differential scanning calorimetry at a scan rate of 10° C. per minute.

In yet another variation of the embodiment, the salt of decitabine is a L-aspartate salt in crystalline form characterized by an X-ray diffraction pattern having diffraction peaks (2θ) at 21.61°, 22.71°, and 23.24°. The salt is further characterized by multiple reversible melting endotherms at 30-100° C., 170-195° C., and 195-250° C., optionally at 86° C., 187° C., and 239° C., as measured by differential scanning calorimetry at a scan rate of 10° C. per minute.

In yet another variation of the embodiment, the salt of decitabine is a maleate salt in crystalline form characterized by an X-ray diffraction pattern having diffraction peaks (2θ) at 20.81°, 27.38°, and 28.23°. The salt is further characterized by multiple reversible melting endotherms at 95-130° C. and 160-180° C., or optionally at 119° C. and 169° C., as measured by differential scanning calorimetry at a scan rate of 10° C. per minute.

In yet another variation of the embodiment, the salt of decitabine is a phosphate salt in crystalline form characterized by an X-ray diffraction pattern having diffraction peaks (2θ) at 17.09°, 21.99°, and 23.21°. The salt is further characterized by a melting endotherm at 130-145° C. as measured by differential scanning calorimetry at a scan rate of 10° C. per minute.

In yet another variation of the embodiment, the salt of decitabine is a L-glutamate salt in crystalline form characterized by an X-ray diffraction pattern having diffraction peaks (2θ) at 13.33°, 21.39°, and 30.99°. The salt is further characterized by multiple reversible melting endotherms at 50-100° C., 175-195° C., and 195-220° C., or optionally at 84° C., 183° C., and 207° C. as measured by differential scanning calorimetry at a scan rate of 10° C. per minute.

In yet another variation of the embodiment, the salt of decitabine is a (+)-L-tartarate salt in crystalline form characterized by an X-ray diffraction pattern having diffraction peaks (2θ) at 7.12°, 13.30°, and 14.22°. The salt is further characterized by multiple reversible melting endotherms at 60-110° C., and 185-220° C., optionally at 91° C., and 203° C., as measured by differential scanning calorimetry at a scan rate of 10° C. per minute.

In yet another variation of the embodiment, the salt of decitabine is a citrate salt in crystalline form characterized by an X-ray diffraction pattern having diffraction peaks (2θ) at 13.31°, 14.23°, and 23.26°. The salt is further characterized by multiple reversible melting endotherms at 30-100° C. and 160-220° C., or optionally at 84° C. and 201° C., as measured by differential scanning calorimetry at a scan rate of 10° C. per minute.

In yet another variation of the embodiment, the salt of decitabine is a L-lactate salt in crystalline form characterized by an X-ray diffraction pattern having diffraction peaks (2θ) at 13.27°, 21.13°, and 23.72°. The salt is further characterized by multiple reversible melting endotherms at 30-100° C. and 160-210° C., or optionally at 84° C. and 198° C., as measured by differential scanning calorimetry at a scan rate of 10° C. per minute.

In yet another variation of the embodiment, the salt of decitabine is a succinate salt in crystalline form characterized by an X-ray diffraction pattern having diffraction peaks (2θ) at 13.30°, 22.59°, and 23.28°. The salt is further characterized by multiple reversible melting endotherms at 50-100° C. and 190-210° C., or optionally at 79° C. and 203° C., as measured by differential scanning calorimetry at a scan rate of 10° C. per minute.

In yet another variation of the embodiment, the salt of decitabine is an acetate salt in crystalline form characterized by an X-ray diffraction pattern having diffraction peaks (2θ) at 7.14°, 14.26°, and 31.25°. The salt is further characterized by multiple reversible melting endotherms at 60-90° C. and 185-210° C., or optionally at 93° C. and 204° C., as measured by differential scanning calorimetry at a scan rate of 10° C. per minute.

In yet another variation of the embodiment, the salt of decitabine is a hexanoate salt in crystalline form characterized by an X-ray diffraction pattern having diffraction peaks (2θ) at 13.27°, 22.54°, and 23.25°. The salt is further characterized by multiple reversible melting endotherms at 60-90° C. and 190-210° C., or optionally at 93° C. and 204° C., as measured by differential scanning calorimetry at a scan rate of 10° C. per minute.

In yet another variation of the embodiment, the salt of decitabine is a butyrate salt in crystalline form characterized by an X-ray diffraction pattern having diffraction peaks (2θ) at 13.28°, 22.57°, and 23.27°. The salt is further characterized by multiple reversible melting endotherms at 40-90° C. and 190-210° C., or optionally at 89° C. and 203° C., as measured by differential scanning calorimetry at a scan rate of 10° C. per minute.

In yet another variation of the embodiment, the salt of decitabine is a propionate salt in crystalline form characterized by an X-ray diffraction pattern having diffraction peaks (2θ) at 13.29°, 22.52°, and 23.27°. The salt is further characterized by multiple reversible melting endotherms at 50-110° C. and 190-210° C., optionally at 94° C. and 204° C., as measured by differential scanning calorimetry at a scan rate of 10° C. per minute.

In yet another embodiment, a salt of azacitidine is provided. The salt of azacitidine is a hydrochloride, mesylate, EDTA, sulfite, L-Aspartate, maleate, phosphate, L-Glutamate, (+)-L-Tartrate, citrate, L-Lactate, succinate, acetate, hexanoate, butyrate, or propionate salt.

According to the embodiment, the salt of azacitidine is a mesylate salt in crystalline form characterized by an X-ray diffraction pattern having diffraction peaks (2θ) at 18.58°, 23.03°, and 27.97°. The salt is further characterized by multiple reversible melting endotherms at 30-80° C., 80-110° C. and 110-140° C. as measured by differential scanning calorimetry at a scan rate of 10° C. per minute.

Also according to the present invention, a method is provided for treating a disease associated with undesirable cell proliferation in a subject. The method comprises administering to the subject in need thereof a pharmaceutically effective amount of a salt of a cytidine analog. The disease may be benign tumors, cancer, hematological disorders, atherosclerosis, insults to body tissue due to surgery, abnormal wound healing, abnormal angiogenesis, diseases that produce fibrosis of tissue, repetitive motion disorders, disorders of tissues that are not highly vascularized, or proliferative responses associated with organ transplants. In particular, the disease is myelodysplastic syndrome, non-small cell lung cancer, or sickle-cell anemia.

The salts of present invention can be formulated in various ways and delivered to a patient suffering from a disease sensitive to the treatment with a cytidine analog via various routes of administration such as intravenous, intramuscular, subcutaneous injection, oral administration and inhalation.

The present invention also provides methods for synthesizing, formulating and manufacturing salts of a cytidine analog.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a DSC plot of decitabine hydrochloride.

FIG. 2 illustrates a DSC plot of decitabine mesylate.

FIG. 3 illustrates a DSC plot of decitabine EDTA.

FIG. 4 illustrates a DSC plot of decitabine L-aspartate.

FIG. 5 illustrates a DSC plot of decitabine maleate.

FIG. 6 illustrates a DSC plot of decitabine L-glutamate.

FIG. 7 illustrates a DSC plot of decitabine sulfite.

FIG. 8 illustrates a DSC plot of decitabine phosphate.

FIG. 9 illustrates a DSC plot of decitabine tartrate.

FIG. 10 illustrates a DSC plot of decitabine citrate.

FIG. 11 illustrates a DSC plot of decitabine L-(+)-lactate.

FIG. 12 illustrates a DSC plot of decitabine succinate.

FIG. 13 illustrates a DSC plot of decitabine acetate.

FIG. 14 illustrates a DSC plot of decitabine hexanoate.

FIG. 15 illustrates a DSC plot of decitabine butyrate.

FIG. 16 illustrates a DSC plot of decitabine propionate.

FIG. 17 illustrates a DSC plot of azacitidine mesylate.

FIG. 18 illustrates a TGA plot of decitabine hydrochloride.

FIG. 19 illustrates a TGA plot of decitabine mesylate.

FIG. 20 illustrates a TGA plot of decitabine EDTA.

FIG. 21 illustrates a TGA plot of decitabine L-aspartate.

FIG. 22 illustrates a TGA plot of decitabine maleate.

FIG. 23 illustrates a TGA plot of decitabine L-glutamate.

FIG. 24 illustrates a TGA plot of decitabine sulfite.

FIG. 25 illustrates a TGA plot of decitabine phosphate.

FIG. 26 illustrates a TGA plot of decitabine tartrate.

FIG. 27 illustrates a TGA plot of decitabine citrate.

FIG. 28 illustrates a TGA plot of decitabine L-(+)-lactate.

FIG. 29 illustrates a TGA plot of decitabine succinate.

FIG. 30 illustrates a TGA plot of decitabine acetate.

FIG. 31 illustrates a TGA plot of decitabine hexanoate.

FIG. 32 illustrates a TGA plot of decitabine butyrate.

FIG. 33 illustrates a TGA plot of decitabine propionate.

FIG. 34 illustrates a TGA plot of azacitidine mesylate.

FIG. 35 illustrates an XRD pattern of decitabine hydrochloride.

FIG. 36 illustrates an XRD pattern of decitabine mesylate.

FIG. 37 illustrates an XRD pattern of decitabine EDTA.

FIG. 38 illustrates an XRD pattern of decitabine L-aspartate.

FIG. 39 illustrates an XRD pattern of decitabine maleate.

FIG. 40 illustrates an XRD pattern of decitabine L-glutamate.

FIG. 41 illustrates an XRD pattern of decitabine sulfite.

FIG. 42 illustrates an XRD pattern of decitabine phosphate.

FIG. 43 illustrates an XRD pattern of decitabine tartrate.

FIG. 44 illustrates an XRD pattern of decitabine citrate.

FIG. 45 illustrates an XRD pattern of decitabine L-(+)-lactate.

FIG. 46 illustrates an XRD pattern of decitabine succinate.

FIG. 47 illustrates an XRD pattern of decitabine acetate.

FIG. 48 illustrates an XRD pattern of decitabine hexanoate.

FIG. 49 illustrates an XRD pattern of decitabine butyrate.

FIG. 50 illustrates an XRD pattern of decitabine propionate.

FIG. 51 illustrates an XRD pattern of azacitidine mesylate.

FIG. 52 illustrates an IR absorbance spectrum of decitabine hydrochloride.

FIG. 53 illustrates an IR absorbance spectrum of decitabine mesylate.

FIG. 54 illustrates an IR absorbance spectrum of decitabine EDTA.

FIG. 55 illustrates an IR absorbance spectrum of decitabine L-aspartate.

FIG. 56 illustrates an IR absorbance spectrum of decitabine maleate.

FIG. 57 illustrates an IR absorbance spectrum of decitabine L-glutamate.

FIG. 58 illustrates an IR absorbance spectrum of decitabine sulfite.

FIG. 59 illustrates an IR absorbance spectrum of decitabine phosphate.

FIG. 60 illustrates an IR absorbance spectrum of decitabine tartrate.

FIG. 61 illustrates an IR absorbance spectrum of decitabine citrate.

FIG. 62 illustrates an IR absorbance spectrum of decitabine L-(+)-lactate.

FIG. 63 illustrates an IR absorbance spectrum of decitabine succinate.

FIG. 64 illustrates an IR absorbance spectrum of decitabine acetate.

FIG. 65 illustrates an IR absorbance spectrum of decitabine hexanoate.

FIG. 66 illustrates an IR absorbance spectrum of decitabine butyrate.

FIG. 67 illustrates an IR absorbance spectrum of decitabine propionate.

FIG. 68 illustrates an IR absorbance spectrum of azacitidine mesylate.

### DETAILED DESCRIPITION OF THE PRESENT INVENTION

The present invention provides salts of cytidine analogs, e.g., decitabine and azacitidine, which can be used as pharmaceuticals for the treatment of various diseases and conditions, such as myelodysplastic syndrome (MDS), non-small cell lung (NSCL) cancer, and sickle-cell anemia. This innovative approach is taken to overcome three major hurdles that have adversely impacted the commercial development of this type of drugs: hydrolytic degradation in aqueous environment; low solubility in most pharmaceutically acceptable solvents; and minimal oral bioavailability.

According to the present invention, the solid state and solution properties of a cytidine analog is modified by salt formation. The inventors believe that salt formation can lead to improved solubility and stability of this type of drugs, such as decitabine and azacitidine. Increased water-solubility can also potentially make the drug entities less toxic. Due to their easier renal clearance they are less likely to accumulate and overload the hepatic microsomes responsible for phase-one and phase-two metabolism. Further more, increased stability can make manufacturing of the drug product more robust and facilitate development of different formulations.

The salts of present invention can be formulated in various ways and delivered to a patient suffering from a disease sensitive to the treatment with a cytidine analog, such as hematological disorders, benign tumors, malignant tumors, restenosis, and inflammatory diseases via various routes of administration such as intravenous, intramuscular, subcutaneous injection, oral administration and inhalation.

The present invention also provides methods for synthesizing, formulating and manufacturing salts of cytidine analogs, and methods for using the salts for treating various diseases and conditions.

The following is a detailed description of the invention and preferred embodiments of the inventive salts, compositions, methods of use, synthesis, formulations and manufacture.

1. Salts of Cytidine Analogs and Derivatives

One aspect of the invention is the salt form of a cytidine analog or derivative, preferably a salt of 5-aza-2′-deoxycytidine (decitabine 1) or 5-azacytidine (azacitidine 2) whose chemical structures are depicted below:

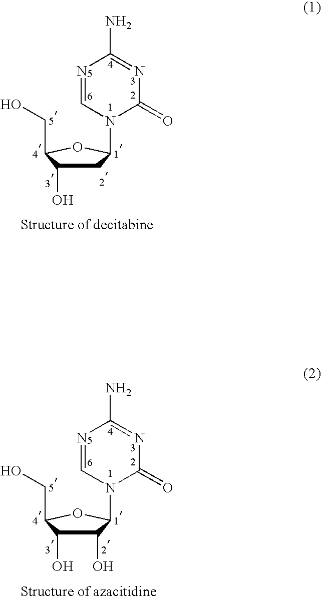

In some embodiments, to ensure sufficient proton transfer from the acid to a basic drug, the newly formed conjugate acid and conjugate base should be weaker than the original acid and basic drug, generally by at least about 2 units weaker than the pKₐ of the drug. Two pKₐ values, 7.61±0.03 and 3.58±0.06, were found for decitabine. In preferred embodiments, an acid with pKₐ lower than about 5, or optionally with pKₐ between 3 and −10, is used to synthesize a salt form of decitabine, as well as a salt form of azacitidine, and other cytidine analogs and derivatives. Examples of suitable acids are listed in Table 1a.

TABLE 1a
Examples of acids that can be used to synthesize a salt form of decitabine,
azacitidine, and other cytidine analogs and derivatives.

| Name | pKₐ₁ | pKₐ₂ | Name | pKₐ₁ | pKₐ₂ |
|---|---|---|---|---|---|
| Perchloric acid | −10 | — | Fumaric acid | 3.03 | 4.38 |
| Hydrobromic acid | −9 | — | Galactaric acid | 3.08 | 3.63 |
| Hydroiodic acid | −9 | — | Hydrofluoric acid | 3.16 | — |
| Hydrochloric acid | −6 | — | Citric acid | 3.13 | 4.76 |
| Naphthalene-1,5-disulfonic acid | −3.37 | −2.64 | D-Glucuronic acid | 3.18 | — |
| Sulfuric acid | −3 | 1.92 | Lactobionic acid | 3.2 | — |
| Ethane-1,2-disulfonic acid | −2.1 | −1.5 | 4-Amino-salicylic acid | 3.25 | 10 |
| Cyclamic acid | −2.01 | — | Glycolic acid | 3.28 | — |
| p-Toluenesulfonic acid | −1.34 | — | D-Glucoheptonic acid | 3.3 | — |
| Thiocyanic acid | −1.33 | — | Nitrous acid | 3.3 | — |
| Nitric acid | −1.32 | — | (−)-L-Pyroglutamic acid | 3.32 | — |
| Methanesulfonic acid | −1.2 | — | DL-Mandelic acid | 3.37 | — |
| Chloric acid | −1.0 | — | (−)-L-Malic acid | 3.46 | 5.10 |
| Chromic acid | −0.98 | 6.50 | Hippuric acid | 3.55 | — |
| Dodecylsulfuric acid | −0.09 | — | Formic acid | 3.75 | — |
| Trichloroacetic acid | 0.52 | — | D-Gluconic acid | 3.76 | — |
| Benzenesulfonic acid | 0.7 | — | DL-Lactic acid | 3.86 | — |
| Iodic | 0.80 | — | Oleic acid | 4 | — |
| Oxalic acid | 1.27 | 4.27 | L-Ascorbic acid | 4.17 | 11.57 |
| 2,2-Dichloro-acetic acid | 1.35 | — | Benzoic acid | 4.19 | — |
| Glycerophosphoric acid | 1.47 | — | Succinic acid | 4.21 | 5.64 |
| 2-Hydroxy-ethanesulfonic acid | 1.66 | — | 4-Acetamido-benzoic acid | 4.3 | — |
| EDTA | 1.70 | 2.60 | Glutaric acid | 4.34 | 5.27 |
| Phosphorous acid | 1.80 | 6.15 | Cinnamic acid | 4.40 | |
| Sulfurous | 1.85 | 7.20 | Adipic acid | 4.44 | 5.44 |
| L-Aspartic | 1.88 | 3.65 | Sebacic acid | 4.59 | 5.59 |
| Maleic acid | 1.92 | 6.23 | (+)-Camphoric acid | 4.72 | 5.83 |
| Phosphoric acid | 1.96 | 7.12 | Acetic acid | 4.76 | — |
| Chlorous acid | 1.98 | — | Hexanoic acid | 4.8 | — |
| Ethanesulfonic acid | 2.05 | — | Butyric acid | 4.83 | — |
| (+)-Camphor-10-sulfonic acid | 2.17 | — | Nicotinic acid | 4.85 | — |
| Glutamic acid | 2.19 | 4.25 | Isobutyric acid | 4.86 | — |
| Alginic acid | >2.4 | — | Propionic acid | 4.87 | — |
| Pamoic acid | 2.51 | — | Decanoic acid | 4.9 | — |
| Glutaric acid | 2.7 | — | Lauric acid | 4.9 | — |
| 1-Hydroxy-2-naphthoic acid | 2.7 | — | Palmitic acid | 4.9 | — |
| Malonic acid | 2.83 | — | Stearic acid | 4.9 | — |
| Gentisic acid | 2.93 | — | Undecylenic acid | 4.9 | — |
| Salicylic acid | 2.97 | — | Octanoic acid | 4.91 | — |
| (+)-L-Tartaric acid | 3.02 | 4.36 | Malic acid | 5.05 | — |

In preferred embodiments, decitabine and azacitidine salts are formed with strong acids (pKₐ<0). In other preferred embodiments, the decitabine salts show improved stability over decitabine free base in near neutral pH solutions. By “near neutral pH” is meant a pH at about 7±1, ±2, or ±3.

In preferred embodiments, salts of some cytidine analogs, e.g., decitabine salts, can show some type of protective ionic complex across the N-5 imine nitrogen and the 6-carbon in aqueous solution. Without being limited to a particular hypothesis, such an ionic complex may shield against nucleophilic attack from surrounding water molecules. The illustration below depicts the formation of a protective ion complex (1a, 1b), hypothesized to form in some preferred embodiments of decitabine salts of the instant invention, e.g., where X is a conjugate base such as chloride, mesylate, or phosphate.

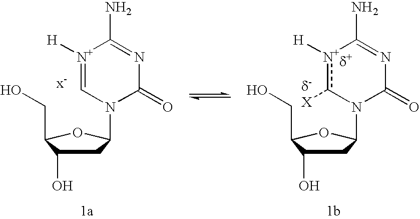

As illustrated, a temporary ionic adduct may form across the 5- and 6-position of decitabine, possibly helping to shield against hydrolytic cleavage in solution.

One embodiment of the invention is the salt form of decitabine synthesized with an acid. Some embodiments include salt forms synthesized with the following acids—HCl, L-lactic, acetic, phosphoric, (+)-L-tartaric, citric, propionic, butyric, hexanoic, L-aspartic, L-glutamic, succinic, EDTA, maleic, and methanesulfonic. Other embodiments include decitabine salts of other common acids. Examples of suitable inorganic acids include, but are not limited to, HBr, HF, HI, nitric, nitrous, sulfuric, sulfurous, phosphorous, perchloric, chloric, and chlorous acid. Examples of suitable carboxylic acids include, but are not limited to, ascorbic, carbonic, and fumaric acid. Examples of suitable sulfonic acids include, but are not limited to, ethanesulfonic, 2-hydroxyethanesulfonic, and toluenesulfonic acid.

Preferably, the molar ratios of acids to decitabine are about 0.01 to about 10 molar equivalents. Preferred embodiments include decitabine salts of strong acids (pKa<0). More preferred embodiments include decitabine hydrochloride (3) and decitabine mesylate (4), illustrated below, which can form in 1:1 molar equivalent (e.g., as determined from elemental analysis).

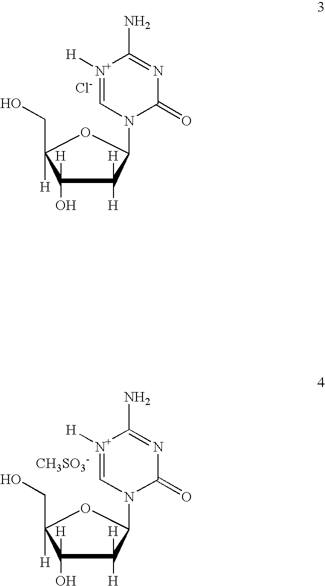

Some preferred embodiments include decitabine salts of moderate acids (0<pKa<3). Preferred salts formed with moderate acids include decitabine EDTA (5), L-aspartate (6), maleate (7) and L-glutamate (8), depicted below:

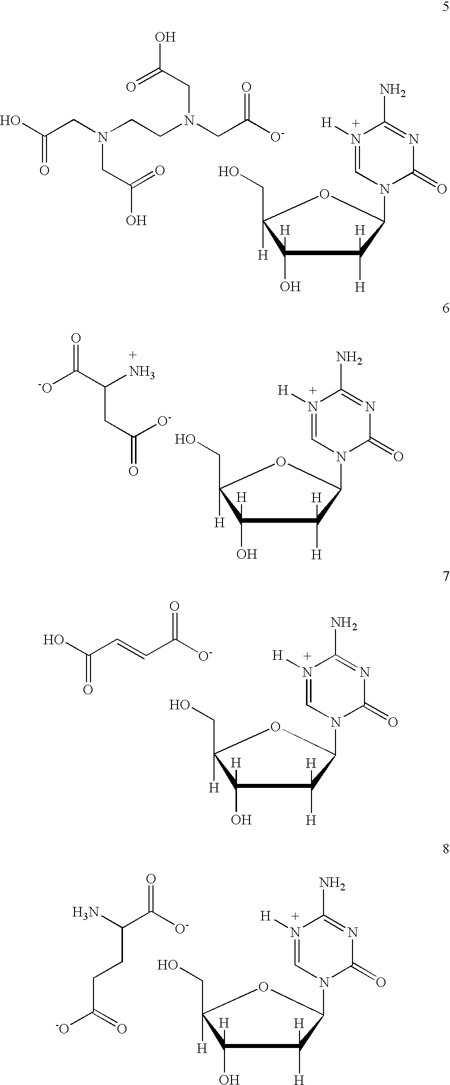

Still other preferred salts formed with moderate acids (0<pKa<3) include decitabine sulfite (9) or decitabine phosphate (10), depicted below:

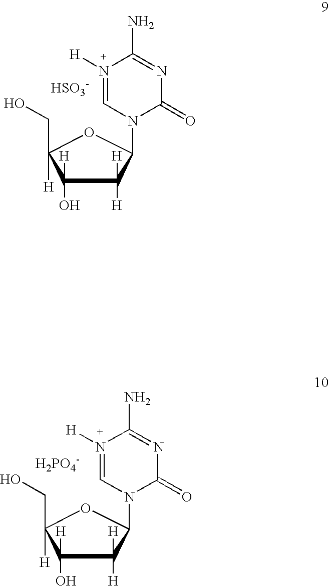

Some embodiments include decitabine salts of weak acids (3<pKa<5). Examples of salts formed with weak acids include decitabine (+)-L-tartrate (11); decitabine citrate (12); decitabine L-Lactate (13); decitabine succinate (14); decitabine acetate (15); decitabine hexanoate (16); decitabine butyrate (17); and decitabine propionate (18), each depicted below:

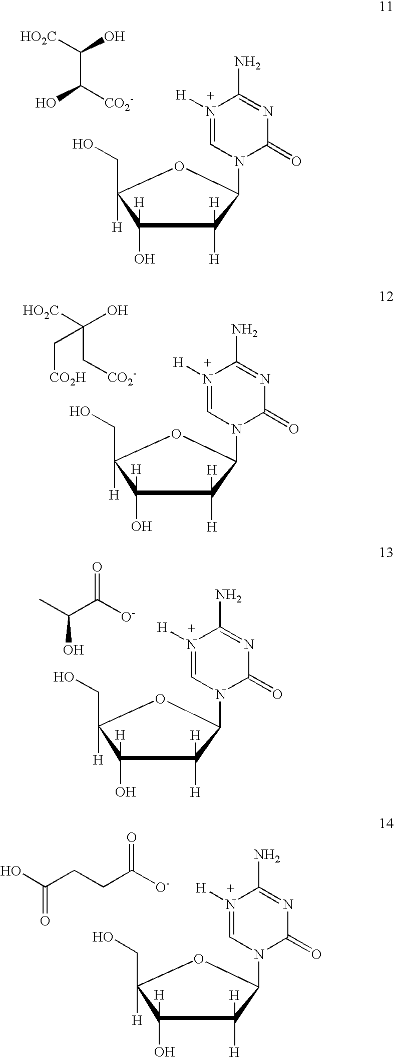

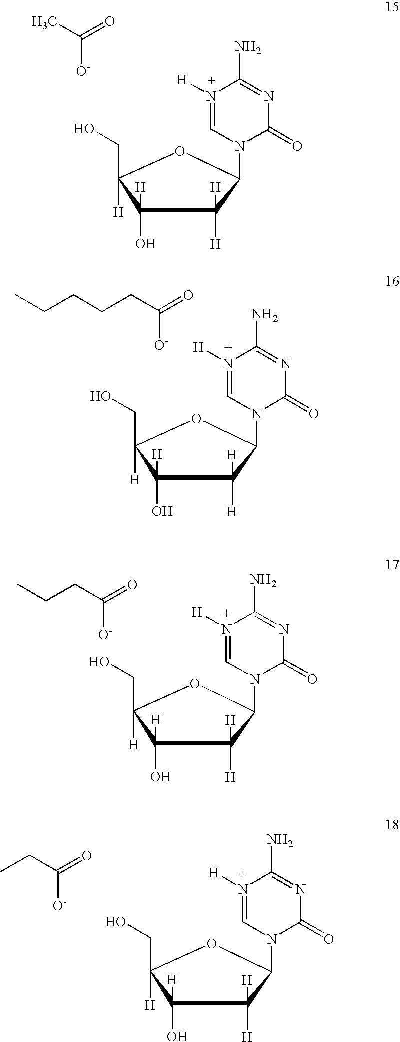

A Second aspect of the invention is a salt form of azacitidine. One embodiment is an azacitidine salt of methanesulfonic acid, e.g. azacitidine mesylate (19), depicted below:

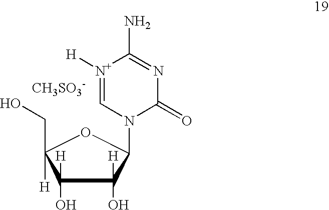

Other embodiments include azacitidine salts of inorganic or organic acids. Examples of suitable inorganic acids include, but are not limited to, HCl, HBr, HF, HI, nitric, nitrous, sulfuric, sulfurous, phosphoric, phosphorous, perchloric, chloric, and chlorous acid. Examples of suitable carboxylic acids include, but are not limited to, acetic, ascorbic, butyric, carbonic, citric, EDTA, fumaric, hexanoic, L-lactic, maleic, propionic, succinic, and (+)-L-tartaric acid. Other suitable acids for forming azacitidine salts include sulfuric and amino acids. Examples of suitable sulfonic acids include, but are not limited to, ethanesulfonic, 2-hydroxyethanesulfonic, and toluenesulfonic acid. Examples of suitable amino acids include, but are not limited to, L-aspartic and L-glutamic acid.

The present invention also embraces isolated salts of cytidine analogs. An isolated salt of a cytidine analog refers to a salt of a cytidine analog which represents at least 10%, preferably 20%, more preferably 50%, or most preferably 80% of the salt of the cytidine analog present in the mixture.

2. Pharmaceutical Formulations of the Present Invention

According to the present invention, the salts of cytosine analogs can be formulated into pharmaceutically acceptable compositions for treating various diseases and conditions.

The pharmaceutically-acceptable compositions of the present invention comprise one or more salts of the invention in association with one or more nontoxic, pharmaceutically-acceptable carriers and/or diluents and/or adjuvants and/or excipients, collectively referred to herein as “carrier” materials, and if desired other active ingredients.

The salts of the present invention are administered by any route, preferably in the form of a pharmaceutical composition adapted to such a route, as illustrated below and are dependent on the condition being treated. The compounds and compositions can be, for example, administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by a catheter or stent), subcutaneously, intraadiposally, intraarticularly, or intrathecally.

The pharmaceutical formulation may optionally further include an excipient added in an amount sufficient to enhance the stability of the composition, maintain the product in solution, or prevent side effects (e.g., potential ulceration, vascular irritation or extravasation) associated with the administration of the inventive formulation. Examples of excipients include, but are not limited to, mannitol, sorbitol, lactose, dextrox, cyclodextrin such as, α-, β-, and γ-cyclodextrin, and modified, amorphous cyclodextrin such as hydroxypropyl-, hydroxyethyl-, glucosyl-, maltosyl-, maltotriosyl-, carboxyamidomethyl-, carboxymethyl-, sulfobutylether-, and diethylamino-substituted α-, β-, and γ-cyclodextrin. Cyclodextrins such as Encapsin® from Janssen Pharmaceuticals or equivalent may be used for this purpose.

For oral administration, the pharmaceutical compositions can be in the form of, for example, a tablet, capsule, suspension or liquid. The pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents. Oral liquid preparations generally are in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For topical use the salts of the present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the salts of the present invention can be presented in liquid or semi-liquid form formulated in hydrophobic or hydrophilic bases as ointments, creams, lotions, paints or powders.

For rectal administration the salts of the present invention can be administered in the form of suppositories admixed with conventional carriers such as cocoa butter, wax or other glyceride.

Alternatively, the salts of the present invention can be in powder form for reconstitution in the appropriate pharmaceutically acceptable carrier at the time of delivery.

The pharmaceutical compositions can be administered via injection. Formulations for parenteral administration can be in the form of aqueous or non-aqueous isotonic sterile injection solutions or suspensions. These solutions or suspensions can be prepared from sterile powders or granules having one or more of the carriers mentioned for use in the formulations for oral administration. The salts can be dissolved in water, polyethylene glycol, propylene glycol, ethanol, corn oil, benzyl alcohol, sodium chloride, and/or various buffers.

In an embodiment, the salt of the present invention can be formulated into a pharmaceutically acceptable composition comprising the compound solvated in non-aqueous solvent that includes glycerin, propylene glycol, polyethylene glycol, or combinations thereof. It is believed that the compound decitabine will be stable in such pharmaceutical formulations so that the pharmaceutical formulations may be stored for a prolonged period of time prior to use.

As discussed above, in current clinical treatment with decitabine, to minimize drug decomposition decitabine is supplied as lyophilized powder and reconstituted in a cold aqueous solution containing water in at least 40% vol. of the solvent, such as WFI, and diluted in cold infusion fluids prior to administration. Such a formulation and treatment regimen suffers from a few drawbacks. First, refrigeration of decitabine in cold solution becomes essential, which is burdensome in handling and economically less desirable than a formulation that can sustain storage at higher temperatures. Second, due to rapid decomposition of decitabine in aqueous solution, the reconstituted decitabine solution may only be infused to a patient for a maximum of 3 hr if the solution has been stored in the refrigerator for less than 7 hr. In addition, infusion of cold fluid can cause great discomfort and pain to the patient, which induces the patient's resistance to such a regimen.

By modifying the solid state and solution properties of cytidine analogs, the pharmaceutical formulations comprising the inventive salts can circumvent the above-listed problems associated with the current clinical treatment with decitabine and azacitidine. The inventive salts can be formulated in aqueous solutions containing water in at least 40% vol. of the solvent, optionally at least 80%, or optionally at least 90% vol. of the solvent. These formulations of the inventive salts are believed to be more chemically stable than the free base form of decitabine or azacitidine formulated in aqueous solutions.

Alternatively, the inventive salts may be formulated in solutions containing less than 40% water in the solvent, optionally less than 20% water in the solvent, optionally less than 10% water in the solvent, or optionally less than 1% water in the solvent. In one variation, the pharmaceutical formulation is stored in a substantially anhydrous form. Optionally, a drying agent may be added to the pharmaceutical formulation to absorb water.

Owing to the enhanced stability, the inventive formulation may be stored and transported at ambient temperature, thereby significantly reducing the cost of handling the drug. Further, the inventive formulation may be conveniently stored for a long time before being administered to the patient. In addition, the inventive formulation may be diluted with regular infusion fluid (without chilling) and administered toga patient at room temperature, thereby avoiding causing patients' discomfort associated with infusion of cold fluid.

In another embodiment, the inventive salt is dissolved in a solution at different concentrations. For example, the formulation may optionally comprise between 0.1 and 200; between 1 and 100; between 1 and 50; between 2 and 50; between 2 and 100; between 5 and 100; between 10 and 100 or between 20 and 100 mg inventive salt per ml of the solution. Specific examples of the inventive salt per solution concentrations include but are not limited to 2, 5, 10, 20, 22, 25, 30, 40 and 50 mg/ml.

In yet another embodiment, the inventive salt is dissolved in a solvent combining glycerin and propylene glycol at different concentrations. The concentration of propylene glycol in the solvent is between 0.1-99.9%, optionally between 1-90%, between 10-80%, or between 50-70%.

In yet another embodiment, the inventive salt is dissolved at different concentrations in a solvent combining glycerin and polyethylene glycol (PEG) such as PEG300, PEG400 and PEG1000. The concentration of polyethylene glycol in the solvent is between 0.1-99.9%, optionally between 1-90%, between 10-80%, or between 50-70%.

In yet another embodiment, the inventive salt is dissolved at different concentrations in a solvent combining propylene glycol, polyethylene glycol and glycerin. The concentration of propylene glycol in the solvent is between 0.1-99.9%, optionally between 1-90%, between 10-60%, or between 20-40%; and the concentration of polyethylene glycol in the solvent is between 0.1-99.9%, optionally between 1-90%, between 10-80%, or between 50-70%.

It is believed that addition of propylene glycol can further improve chemical stability, reduce viscosity of the formulations and facilitate dissolution of the inventive salt in the solvent.

The pharmaceutical formulation may further comprise an acidifying agent added to the formulation in a proportion such that the formulation has a resulting pH between about 4 and 8. The acidifying agent may be an organic acid. Examples of organic acid include, but are not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid. The acidifying agent may also be an inorganic acid, such as hydrochloric acid, sulphuric acid, phosphoric acid, and nitric acid.

It is believed that adding an acidifying agent to the formulation to maintain a relatively neutral pH (e.g., within pH 4-8) facilitates ready dissolution of the inventive compound in the solvent and enhances long-term stability of the formulation. In alkaline solution, there is a rapid reversible decomposition of decitabine to N-(formylamidino)-N′-β-D-2-deoxyribofuiranosylurea, which decomposes irreversibly to form 1-β-D-2′-deoxyribofuranosyl-3-guanylurea. The first stage of the hydrolytic degradation involves the formation of N-amidinium-N′-(2-deoxy-β-D-erythropentofuranosyl)urea formate (AUF). The second phase of the degradation at an elevated temperature involves formation of guanidine. In acidic solution, N-(formylamidino)-N′-β-D-2-deoxyribofuranosylurea and some unidentified compounds are formed. In strongly acidic solution (at pH<2.2) 5-azacytosine is produced. Thus, maintaining a relative neutral pH may be advantageous for the formulation comprising the inventive salt.

In a variation, the acidifying agent is ascorbic acid at a concentration of 0.01-0.2 mg/ml of the solvent, optionally 0.04-0.1 mg/ml or 0.03-0.07 mg/ml of the solvent.

The pH of the pharmaceutical formulation may be adjusted to be between pH 4 and pH 8, preferably between pH 5 and pH 7, and more preferably between pH 5.5 and pH 6.8.

The pharmaceutical formulation is preferably at least 80%, 90%, 95% or more stable upon storage at 25° C. for 7, 14, 21, 28 or more days. The pharmaceutical formulation is also preferably at least 80%, 90%, 95% or more stable upon storage at 40° C. for 7, 14, 21, 28 or more days.

In one embodiment, the. pharmaceutical formulation of the present invention is prepared by taking glycerin and dissolving the inventive compound in the glycerin. This may be done, for example, by adding the inventive salt to the glycerin or by adding the glycerin to the inventive salt. By their admixture, the pharmaceutical formulation is formed.

Optionally, the method further comprises additional steps to increase the rate at which the inventive salt is solvated by the glycerin. Examples of additional steps that may be performed include, but are nor limited to, agitation, heating, extension of solvation period, and application of micronized inventive compound and the combinations thereof.

In one variation, agitation is applied. Examples of agitation include, but are nor limited to, mechanical agitation, sonication, conventional mixing, conventional stirring and the combinations thereof. For example, mechanical agitation of the formulations may be performed according to manufacturer's protocols by Silverson homogenizer manufactured by Silverson Machines Inc., (East Longmeadow, Mass.).

In another variation, heat may be applied. Optionally, the formulations may be heated in a water bath. Preferably, the temperature of the heated formulations may be less than 70° C., more preferably, between 25° C. and 40° C. As an example, the formulation may be heated to 37° C.

In yet another variation, the inventive salt is solvated in glycerin over an extended period of time.

In yet another variation, a micronized form of the inventive salt may also be employed to enhance solvation kinetics. Optionally, micronization may be performed by a milling process. As an example, micronization may be performed according to manufacturer's protocols by jet milling process performed by Malvern Mastersizer, Mastersizerusing an Air Jet Mill, manufactured by Micron Technology Inc.(Boise, Id.).IncFluid Energy Aljet Inc. (Boise, Id.Telford, Pa.).

Optionally, the method further comprises adjusting the pH of the pharmaceutical formulations by commonly used methods. In one variation, pH is adjusted by addition of acid, such as ascorbic acid, or base, such as sodium hydroxide. In another variation, pH is adjusted and stabilized by addition of buffered solutions, such as solution of (Ethylenedinitrilo) tetraacetic acid disodium salt (EDTA). As decitabine and azacitidine are known to be pH-sensitive, adjusting the pH of the pharmaceutical formulations to approximately pH 7 may increase the stability of therapeutic component.

Optionally, the method further comprises separation of non-dissolved inventive salt from the pharmaceutical formulations. Separation may be performed by any suitable technique. For example, a suitable separation method may include one or more of filtration, sedimentation, and centrifugation of the pharmaceutical formulations. Clogging that may be caused by non-dissolved particles of the inventive compound, may become an obstacle for administration of the pharmaceutical formulations and a potential hazard for the patient. The separation of non-dissolved inventive compound from the pharmaceutical formulations may facilitate administration and enhance safety of the therapeutic product.

Optionally, the method further comprises sterilization of the pharmaceutical formulations. Sterilization may be performed by any suitable technique. For example, a suitable sterilization method may include one or more of sterile filtration, chemical, irradiation, heat filtration, and addition of a chemical disinfectant to the pharmaceutical formulation.

Optionally, the method may further comprise adding one or more members of the group selected from drying agents, buffering agents, antioxidants, stabilizers, antimicrobials, and pharmaceutically inactive agents. In one variation, antioxidants such as ascorbic acid, ascorbate salts and mixtures thereof may be added. In another variation stabilizers like glycols may be added.

3. Vessels or Kits Containing Inventive Salts or Formulations Thereof

The inventive salts or their formulations described in this invention may be contained in a sterilized vessel such as syringe bottles, and glass vials or ampoules of various sizes and capacities. The sterilized vessel may optionally contain solid salt in a form of powder or crystalline, or its solution formulation with a volume of 1-50 ml, 1-25 ml, 1-20 ml or 1-10 ml. Sterilized vessels enable maintain sterility of the pharmaceutical formulations, facilitate transportation and storage, and allow administration of the pharmaceutical formulations without prior sterilization step.

The present invention also provides a kit for administering the inventive compound to a host in need thereof. In one embodiment, the kit comprises the inventive salt in a solid, preferably powder form, and a liquid diluent that comprises water, glyercin, propylene glycol, polyethylene glycol, or combinations thereof. Mixing of the solid salt and the diluent preferably results in the formation of a pharmaceutical formulation according to the present invention. For example, the kit may comprise a first vessel comprising the inventive salt in a solid form; and a vessel container comprising a diluent that comprises water; wherein adding the diluent to the solid inventive compound results in the formation of a pharmaceutical formulation for administering the inventive salt. Mixing the solid the inventive salt and diluent may optionally form a pharmaceutical formulation that comprises between 0.1 and 200 mg the inventive salt per ml of the diluent, optionally between 0.1 and 100, between 2 mg and 50 mg, 5 mg and 30 mg, between 10 mg and 25 mg per ml of the solvent.

In one embodiment, the diluent is a combination of propylene glycol and glycerin, wherein the concentration of propylene glycol in the solvent is between 0.1-99.9%, optionally between 1-90%, between 10-60%, or between 20-40%.

According to the embodiment, the diluent is a combination of polyethylene glycol and glycerin, wherein the concentration of polyethylene glycol in the solvent is between 0.1-99.9%, optionally between 1-90%, between 10-60%, or between 20-40%.

Also according to the embodiment, the diluent is a combination of propylene glycol, polyethylene glycol and glycerin, wherein the concentration of propylene glycol in the solvent is between 0.1-99.9%, optionally between 1-90%, between 10-60%, or between 20-40%; and the concentration of polyethylene glycol in the solvent is between 0.1-99.9%, optionally between 1-90%, between 10-60%, or between 20-40%.

The diluent also optionally comprises 40%, 20%, 10%, 5%, 2% or less water. In one variation, the diluent is anhydrous and may optionally fturther comprise a drying agent. The diluent may also optionally comprise one or more drying agents, glycols, antioxidants and/or antimicrobials.

The kit may optionally flurther include instructions. The instructions may describe how the solid salt and the diluent should be mixed to form a pharmaceutical formulation. The instructions may also describe how to administer the resulting pharmaceutical formulation to a patient. It is noted that the instructions may optionally describe the administration methods according to the present invention.

The diluent and the inventive salt may be contained in separate vessels. The vessels may come in different sizes. For example, the vessel may comprise between 1 and 50, 1 and 25, 1 and 20, or 1 and 10 ml of the diluent.

The pharmaceutical formulations provided in vessels or kits may be in a form that is suitable for direct administration or may be in a concentrated form that requires dilution relative to what is administered to the patient. For example, pharmaceutical formulations, described in this invention, may be in a form that is suitable for direct administration via infusion.

The methods and kits described herein provide flexibility wherein stability and therapeutic effect of the pharmaceutical formulations comprising the inventive compound may be further enhanced or complemented.

4. Methods for Administrating Inventive Salts and Formulations Thereof

The salts/formulations of the present invention can be administered by any route, preferably in the form of a pharmaceutical composition adapted to such a route, as illustrated below and are dependent on the condition being treated. The compounds or formulations can be, for example, administered orally, parenterally, topically, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or co-administered in slow release dosage forms.

The salts/formulations of this invention may be administered or co-administered in any conventional dosage form Co-administration in the context of this invention is defined to mean the administration of more than one therapeutic agent in the course of a coordinated treatment to achieve an improved clinical outcome. Such co-administration may also be coextensive, that is, occurring during overlapping periods of time.

The inventive salts/formulations may be administered into a host such as a patient at a dose of 0.1-1000 mg/m², optionally 1-200 mg/m², optionally 1-150 mg/m², optionally 1-100 mg/m², optionally 1-75 mg/m², optionally 1-50 mg/m², optionally 1-40 mg/m², optionally 1-30 mg/M², optionally 1-20 mg/m², or optionally 5-30 mg/m².

For example, the salts of the present invention may be supplied as sterile powder for injection, optionally together with buffering salt such as potassium dihydrogen and pH modifier such as sodium hydroxide. This formulation is preferably stored at 2-8° C., which should keep the drug stable for at least 2 years. This powder formulation may be reconstituted with 10 ml of sterile water for injection. This solution may be further diluted with infusion fluid known in the art, such as 0.9% sodium chloride injection, 5% dextrose injection and lactated ringer's injection. It is preferred that the reconstituted and diluted solutions be used within 4-6 hours for delivery of maximum potency.

In a preferred embodiment, the inventive salts/formulations is administered to a patient by injection, such as subcutaneous injection, bolus i.v. injection, continuous i.v. infusion and i.v. infusion over 1 hour. Optionally the inventive compound/composition is administered to a patient via an 1-24 hour i.v. infusion per day for 3-5 days per treatment cycle at a dose of 0.1 -1000 mg/m² per day, optionally at a dose of 1-200 mg/M² per day, optionally at a dose of 1-150 mg/m² per day, optionally at a dose of 1-100 mg/M² per day, optionally at a dose of 2-50 mg/m² per day, optionally at a dose of 10-30 mg/m² per day, or optionally at a dose of 5-20 mg/M² per day,

For decitabine or azacitidine, the dosage below 50 mg/m² is considered to be much lower than that used in conventional chemotherapy for cancer. By using such a low dose of the analog/derivative of decitabine or azacitidine, transcriptional activity of genes silenced in the cancer cells by aberrant methylation can be activated to trigger downstream signal transduction, leading to cell growth arrest, differentiation and apoptosis, which eventually results in death of these cancer cells. This low dosage, however, should have less systemic cytotoxic effect on normal cells, and thus have fewer side effects on the patient being treated.

The pharmaceutical formulations may be co-administered in any conventional form with one or more member selected from the group comprising infusion fluids, therapeutic compounds, nutritious fluids, anti-microbial fluids, buffering and stabilizing agents.

As described above, the inventive salts can be formulated in a liquid form by solvating the inventive compound in a non-aqueous solvent such as glycerin. The pharmaceutical liquid formulations provide the further advantage of being directly administrable, (e.g., without further dilution) and thus can be stored in a stable form until administration. Further, because glycerin can be readily mixed with water, the formulations can be easily and readily further diluted just prior to administration. For example, the pharmaceutical formulations can be diluted with water 180, 60, 40, 30, 20, 10, 5, 2, 1 minute or less before administration to a patient.

Patients may receive the pharmaceutical formulations intravenously. The preferred route of administration is by intravenous infusion. Optionally, the pharmaceutical formulations of the current invention may be infused directly, without prior reconstitution.

In one embodiment, the pharmaceutical formulation is infused through a connector, such as a Y site connector, that has three arms, each connected to a tube. As an example, Baxter® Y-connectors of various sizes can be used. A vessel containing the pharmaceutical formulation is attached to a tube further attached to one arm of the connector. Infusion fluids, such as 0.9% sodium chloride, or 5% dextrose, or 5% glucose, or Lactated Ringer's, are infused through a tube attached to the other arm of the Y-site connector. The infusion fluids and the pharmaceutical formulations are mixed inside the Y site connector. The resulting mixture is infused into the patient through a tube connected to the third arm of the Y site connector. The advantage of this administration approach over the prior art is that the inventive compound is mixed with infusion fluids before it enters the patient's body, thus reducing the time when decomposition of the cytidine analog may occur due to contact with water. For example, the inventive compound is mixed less than 10, 5, 2 or 1 minutes before entering the patient's body.

Patients may be infused with the pharmaceutical formulations for 1, 2, 3, 4, 5 or more hours, as a result of the enhanced stability of the formulations. Prolonged periods of infusion enable flexible schedules of administration of therapeutic formulations.

Alternatively or in addition, speed and volume of the infusion can be regulated according to the patient's needs. The regulation of the infusion of the pharmaceutical formulations can be performed according to existing protocols.

The pharmaceutical formulations may be co-infused in any conventional form with one or more member selected from the group comprising infusion fluids, therapeutic compounds, nutritious fluids, anti-microbial fluids, buffering and stabilizing agents. Optionally, therapeutic components including, but are not limited to, anti-neoplastic agents, alkylating agents, agents that are members of the retinoids superfamily, antibiotic agents, hormonal agents, plant-derived agents, biologic agents, interleukins, interferons, cytokines, immuno-modulating agents, and monoclonal antibodies, may be co-infused with the inventive formulations.

Co-infusion in the context of this invention is defined to mean the infusion of more than one therapeutic agents in a course of coordinated treatment to achieve an improved clinical outcome. Such co-infusion may be simultaneous, overlapping, or sequential. In one particular example, co-infusion of the pharmaceutical formulations and infusion fluids may be performed through Y-type connector.

The pharmokinetics and metabolism of intravenously administered the pharmaceutical formulations resemble the pharmokinetics and metabolism of intravenously administered the inventive salt.

In humans, decitabine displayed a distribution phase with a half-life of 7 minutes and a terminal half-life on the order of 10-35 minutes as measured by bioassay. The volume of distribution is about 4.6 L/kg. The short plasma half-life is due to rapid inactivation of decitabine by deamination by liver cytidine deaminase. Clearance in humans is high, on the order of 126 mL/min/kg. The mean area under the plasma curve in a total of 5 patients was 408 μg/h/L with a peak plasma concentration of 2.01 μM. In patients decitabine concentrations were about 0.4 μg/ml (2 μM) when administered at 100 mg/m² as a 3-hour infusion. During a longer infusion time (up to 40 hours) plasma concentration was about 0.1 to 0.4 μg/mL. With infusion times of 40-60 hours, at an infusion rate of 1 mg/kg/h, plasma concentrations of 0.43-0.76 μg/mL were achieved. The steady-state plasma concentration at an infusion rate of 1 mg/kg/h is estimated to be 0.2-0.5 μg/mL. The half-life after discontinuing the infusion is 12-20 min. The steady-state plasma concentration of decitabine was estimated to be 0.31-0.39 μg/mL during a 6-hour infusion of 100 mg/m². The range of concentrations during a 600-mg/m² infusion was 0.41-16 μg/mL. Penetration of decitabine into the cerebrospinal fluid in man reaches 14-21% of the plasma concentration at the end of a 36-hour intravenous infusion. Urinary excretion of unchanged decitabine is low, ranging from less than 0.01% to 0.9% of the total dose, and there is no relationship between excretion and dose or plasma drug levels. High clearance values and a total urinary excretion of less than 1% of the administered dose suggest that decitabine is eliminated rapidly and largely by metabolic processes.

Owing to their enhanced stability in comparison with the free base form of decitabine or azacitidine, the inventive salts/compositions can enjoy longer shelf life when stored and circumvent problems associated with clinical use of decitabine or azacitidine. For example, the inventive salts may be supplied as lyophilized powder, optionally with an excipient (e.g., cyclodextrin), acid (e.g., ascorbic acid), alkaline (sodium hydroxide), or buffer salt (monobasic potassium dihydrogen phosphate). The lyophilized powder can be reconstituted with sterile water for injection, e.g., i.v., i.p., i.m., or subcutaneously. Optionally, the powder can be reconstituted with aqueous or non-aqueous solvent comprising a water miscible solvent such as glycerin, propylene glycol, ethanol and PEG. The resulting solution may be administered directly to the patient, or diluted further with infusion fluid, such as 0.9% Sodium Chloride; 5% Dextrose; 5% Glucose; and Lactated Ringer's infusion fluid.

The inventive salts/formulations may be stored under ambient conditions or in a controlled environment, such as under refrigeration (2-8° C.; 36-46° F.). Due to their superior stability in comparison with decitabine salts/formulations can be stored at room temperature, reconstituted with injection fluid, and administered to the patient without prior cooling of the drug solution.

In addition, due to their enhanced chemical stability, the inventive compound/composition should have a longer plasma half-life compared to that of decitabine. Thus, the inventive compound/composition may be administered to the patient at a lower dose and/or less frequently than that for decitabine or azacitidine.

5. Indications for Inventive Salts or Formulations Thereof

The inventive salts/formulations described herein have many therapeutic and prophylactic uses. In a preferred embodiment, the salt forms of cytidine analogs and derivatives, including salt forms of decitabine and azacitidine, are used in the treatment of a wide variety of diseases that are sensitive to the treatment with a cytidine analog or derivative, such as the free base form of decitabine or azacitidine. Preferable indications that may be treated using the inventive salts/formulations include those involving undesirable or uncontrolled cell proliferation. Such indications include benign tumors, various types of cancers such as primary tumors and tumor metastasis, restenosis (e.g. coronary, carotid, and cerebral lesions), hematological disorders, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor cells become undifferentiated, do not respond to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

In some embodiments, the salts of the instant invention are used to treat blood disorders, including inherited blood disorders and/or disorders where hemoglobin is defective, e.g., sickle cell anemia. In some embodiments, the salts of the instant invention can be used to treat cancer, including leukemia, pre-leukemia, and other bone marrow-related cancers, e.g., myelodysplatic syndrome (MDS)); as well as lung cancer, e.g., non-small cell lung cancer (NSCL). NSCL can include epidermoid or squamous carcinnoma, adenocarcinoma, and large cell carcinoma. MDS can include refractory anemia, refractory anemia with ringed sideroblasts, refractory anemia with excess blasts, refractory anemia with excess blasts in transformation, and chronic myelomonocytic leukemia.

The present invention provides methods, pharmaceutical compositions, and kits for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying disorder being treated. For example, in patient with sickle cell anemia, therapeutic benefit includes eradication or amelioration of the underlying sickle cell anemia. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, a salt of the present invention provides therapeutic benefit not only when sickle cell anemia is eradicated, but also when an improvement is observed in the patient with respect to other disorders or discomforts that accompany sickle cell anemia, like hand-foot syndrome, fatigue, and or the severity or duration of pain experienced during a crisis (painful episode). Similarly, salts of the present invention can provide therapeutic benefit in ameliorating symptoms associated with cancers, e.g., MDS or NSCL, including anemia, bruising, persistent infections, the size of a lung tumor, and the like.

For prophylactic benefit, a salt of the invention may be administered to a patient at risk of developing a cancer or blood disorder, or to a patient reporting one or more of the physiological symptoms of such a condition, even though a diagnosis of the condition may not have been made.

If necessary or desirable, the salt may be administered in combination with other therapeutic agents. The choice of therapeutic agents that can be co-administered with the compounds and compositions of the invention will depend, in part, on the condition being treated. Examples of other therapeutic agents include, but are not limited to, anti-neoplastic agents, alkylating agents, agents that are members of the retinoids superfamily, antibiotic agents, hormonal agents, plant-derived agents, biologic agents, interleukins, interferons, cytokines, immuno-modulating agents, and monoclonal antibodies. For example, in the case of sickle cell anemia, a salt of the instant invention may be administered with antibiotics and/or hydroxyurea; in the case of MDS or NSCL, a salt of the instant invention may be administered with a chemotherapeutic agent.

Pharmaceutical compositions suitable for use in the present invention include compositions wherein the active ingredients are present in an effective amount, i.e., in an amount effective to achieve therapeutic and/or prophylactic benefit in a condition being treated, including, e.g., a blood disorder, such as sickle cell anemia, MDS, and/or a cancer such as NSCL.

### EXAMPLES

The following examples are intended to illustrate details of the invention, without thereby limiting it in any manner.

1. Synthesis of Salts of Cytidine Analogs

1) Decitabine Salt Formation

In some embodiments of the present invention, preparation of decitabine salts includes stirring a mixture of decitabine and acid (e.g., an acid included in Table 1a) in solvent(s) (e.g., a solvent(s) listed in Table 1b) at −70 to 100° C. for 0 to 24 hours, allowing crystallization at −70 to 25° C., and performing filtration and purification by recrystallization from solvent(s).

TABLE 1b
Examples of solvent(s) that can be used for preparation of salts.

| | Solubility of Decitabine free |
|---|---|
| Solvent | base (mg/mL) |
| Acetone | <1 |
| Acetonitrile | <1 |
| Acetonitrile:Water (1:1) | 22 |
| 2-Butanone | <1 |
| Chloroform | <1 |
| Dichloromethane | <1 |
| Dichloromethane:Ethanol (1:1) | <1 |
| Dichloromethane:Methanol (1:1) | >1 |
| Diethylamine | <1 |
| N,N-Dimethylformamide | 5 |
| 1,4-Dioxane | <2 |
| Ethanol:Water (1:1) | 3 |
| Ethyl Acetate | <1 |
| Ethyl Ether | <1 |
| 1,1,1,3,3,3-Hexafluoro-2-propanol | 18 |
| Hexanes | <1 |
| Methanol | 2 |
| Methanol:2,2,2-Trifluoroethanol (1:1) | >1 |
| Methanol:Water (1:1) | 4 |
| Methyl Sulfide | <1 |
| Methyl Sulfoxide | 37 |
| Nitromethane | <1 |
| 2-Propanol | <1 |
| Tetrahydrofuran | <1 |
| Toluene | <1 |
| 1,1,1-Trichloroethane | <1 |
| 2,2,2-Trifluoroethanol | 2 |
| 2,2,2-Trifluoroethanol:Water (9:1) | 5 |
| Water | 8 |

In some embodiments, decitabine salts were prepared from strong acids. In one embodiment, for example, decitabine hydrochloride (3), depicted above, was prepared by suspending decitabine (0.25 g, 3.7 mmol) in methanol (40 mL) in a round bottom flask (100-mL). The mixture was gently stirred at 22° C. HCl gas (not less than 2-fold excess) was bubbled into the stirred methanol solution until complete dissolution was reached. The solution was concentrated to ⅓ volume, flushed with nitrogen, corked with a rubber septum and allowed to crystallize (0° C.) for NLT 12 h. The first crop of crystalline product was filtered, rinsed with anhydrous ether (5 mL) and dried in vacuo for NLT 12 h. The filtrate was poured back into the 50 mL Erlenmeyer flask, and enough anhydrous ether was added to a cloudy point. The solution was flushed with nitrogen, corked with a rubber septum and allowed to crystallize (0° C.) for NLT 12 h. The second crop of crystalline product was filtered, rinsed with anhydrous ether (40 mL) and dried in vacuo for NLT 12 h.

In one embodiment, for example, decitabine mesylate (4), depicted above, was prepared by suspending decitabine (1.0 g, 3.7 mmol) in methanol (80 mL) in a round bottom flask (250-mL). The solution was flushed with nitrogen gas, corked with a rubber septum, and was gently stirred for 10 minutes at ambient temperature. Methanesulfonic acid (4.0 mL) was injected through the rubber septum slowly, and the mixture was gently stirred for 1 h. The suspension of decitabine immediately disappeared and the mixture became clear before decitabine mesylate recrystallized. The crystals were allowed to completely crystallize (0° C.) for NLT 4 h. The product was thoroughly washed with MeOH (50 mL) during filtration and dried in vacuo for NLT 12 h.

Decitabine salts were also prepared from moderate acids. In some embodiments, for example, decitabine EDTA (5), L-aspartate (6), maleate (7) or L-glutamate (8), depicted above, can be prepared by the following procedure. Ethylenediaminetetraacetic acid (EDTA, 1.409 g, 4.8 mmol), L-Aspartic acid (641 mg), maleic acid (610 mg, 5.3 mmol) or L-glutamic acid (709 mg) was weighed in a 250 ml round bottom flask before adding methanol (100 mL) and decitabine (1.0 g), and the mixture was stirred at 50° C. for 1 hr or longer until the solution was clear. The filtrate was concentrated to about ½ volume to allow crystallization to occur. The solution was flushed with nitrogen, corked with a rubber septum and allowed to crystallize (0° C.) for NLT 4 hrs. The first crop of crystalline product was filtered and dried in vacuo for NLT 12 hrs. In methanol, decitabine formed 1:1 molar equivalent with EDTA (5), 1:1.5 with L-aspartate (6), 0.78 molar equivalent of maleate (7), and 1:1.5 with L-glutamate (8) (see also Table 2 below).

In some further embodiments, for example, decitabine sulfite (9) or phosphate (10), depicted above, was prepared by suspending decitabine (1.0 g, 3.7 mmol) in methanol (80 mL) in a round bottom flask (250 mL). The solution was flushed with nitrogen gas, corked with a rubber septum, and was gently stirred for 10 minutes at ambient temperature. Sulfurous acid (4.0 mL) or phosphoric acid (0.8 mL) was injected through the rubber septum slowly, and the mixture was gently stirred for 1 hr. The suspension of decitabine disappeared and the mixture became clear before decitabine salt recrystallized. The crystals were allowed to completely crystallize (0° C.) for NLT 4 hrs. The product was thoroughly washed with MeOH (50 mL) during filtration and dried in vacuo for NLT 12 hr. In methanol, decitabine formed 1:1 molar equivalent with sulfite (9) and phosphate (10) (see also Table 2 below).

In still some embodiments, decitabine salts were prepared from weak acids (3.0<pKₐ<5). For example, decitabine salts of (+)-L-tartaric, citric, L-lactic, succinic, acetic, hexanoic, butyric, or propionic acid (11-18, respectively, depicted above) were prepared by the following procedure: Decitabine (1.0 g, 4.4 mmol) was suspended in methanol (50 mL) in a round bottom flask (50 mL) and flushed and closed with nitrogen before adding acid (liquid acid: 0.4-4.4 mL; solid acid: 2-5 g) and each was heated in a sonicator at 30-55° C. until complete dissolution. If after 30 minutes complete dissolution hadn't been achieved, more methanol (5 mL) was added every 10 minutes. The solution was allowed to cool to 23° C. and then stored at 0° C. for NLT 12 hrs. The first crop of crystalline product was filtered and dried in vacuo for NLT 12 hr.

Decitabine salts prepared from weak acids (3.0<pKₐ<5) showed less robust results. For example, in methanol, decitabine does not readily formed 1:1 molar equivalent with (+)-L-tartaric, citric, L-lactic, succinic, acetic, hexanoic, butyric, or propionic acid to form the corresponding salts (11-18, respectively, depicted above). Instead, varying ratios of acids, from 0.03 to 0.19 molar equivalents, were obtained (see also Table 2 below), which may indicate that there was partial salt formation. However, this does not necessary mean that 1:1 molar equivalent salts of these weak acids can not be prepared with other solvents.

2) Azacitidine Salt Formation

The synthesis techniques described herein for decitabine salts can also be adapted for preparation of the corresponding azacitidine salts. Analogous salts of azacitidine can also be prepared from acids used in preparation of decitabine salts. For example, in some embodiments of the present invention, preparation of azacitidine salts includes stirring a mixture of azacitidie and acid (e.g., an acid included in Table 1a).

For example, azacitidine mesylate (19, depicted above) is an azacitidine salt formed with the strong acid methanesulfonic acid. In some embodiments, azacitidine mesylate (19) was prepared by suspending azacitidine (0.5 g, 2.0 mmol) in methanol (40 mL) in a round bottom flask (100 mL). The solution was flushed with nitrogen gas, corked with a rubber septum, and was gently stirred for 10 minutes at ambient temperature. Methanesulfonic acid (2.0 mL) was injected through the rubber septum slowly, and the mixture was gently stirred for 1 h. The suspension of decitabine immediately disappeared and the mixture became clear. The volume of the mixture was reduced by half in vacuo, and azacitidine mesylate crystals were allowed to completely crystallize (0° C.) for NLT 4 h. The product was thoroughly washed with MeOH (40 mL) during filtration and dried in vacuo for NLT 12 h. Azacitidine can readily form 1:1 molar equivalent mesylate salt (19).

2. Solubility and Stability of Decitabine and Azacitidine Salts

Table 2 shows the rate of dissolution and total solubility, as well as other selected properties, for some embodiments of the instant invention compared to free decitabine and free azacitidine. Dissolution rate is based on the time it takes for 1.0 mg of sample to dissolve in water. Dissolution rates for most embodiments, e.g., most decitabine salts, are superior to that of the free base. For example, decitabine hydrochloride (3) (1 second with mixing) and decitabine mesylate (4) (3 seconds with sonication) salts are superior to decitabine free base (1) (3 minutes with sonication). Without being limited to a particular hypothesis, faster rates of dissolution may reduce hydrolytic degradation during manufacture, as well as reducing reconstitution time for powder forms. The rate of dissolution for azacitidine mesylate (19), however, was surprisingly found to be less than the free azacitidine base (2). That is, as shown in Table 2, the dissolution rate for azacitidine mesylate salt (19) (1 minute sonication) is slower than that for azacitidine free base (2) (3 second mixing).

Apparent total solubility was determined by successively adding 5 mg of a sample to a 5-mL vial containing 1.0 mL of deionized water and sonicating the mixture for 1 minute. Additional sample was added in 5-mg increments and sonication for 1 min was repeated until a suspension formed. Total solubilities of most decitabine salt forms are better than or at least as good as decitabine free base. Apparent total solubility for decitabine hydrochloride (3) (280 mg/mL) and decitabine mesylate (4) (195 mg/mL) salts, which is equivalent to 241 mg/mL and 137 mg/mL of free base, respectively, is substantially higher than decitabine free base (1) (8-10 mg/mL). Solubility for 1:1 molar ratio salts such as decitabine-HCl and decitabine-mesylate, for example, increases the solubility of decitabine by more than 10-fold. Similarly, decitabine sulfite (9) and decitabine phosphate (10) show solubilities of 80 mg/mL and 50 mg/mL, respectively, or equivalent to 59 mg/mL and 35 mg/mL of free decitabine base respectively. One of skill in the art will recognize, however, that for some other decitabine salts, the total solubility measurements may not be representative of their 1:1 free base: acid molar ratio equivalents.

With respect to azacitidine mesylate (19), while its rate of dissolution was surprisingly found to be less than that of free azacitidine base (2), as noted above, the apparent total solubility is greatly enhanced, i.e., 205 mg/mL for the salt (19) (equivalent to 137 mg/mL of free azacitidine base) compared with 14 mg/mL for azacitidine free base (2).

TABLE 2
Summary of selected properties of decitabine and azacitidine salts

| | | | | Dissolution | Total |
|---|---|---|---|---|---|
| | | C₈H₁₂N₄O₄._{—} | | In water | Solubility |
| Compound # | Salt | Acid Molar Ratio^{#} | Appearance | (1.0 mg/mL) | (mg/mL) |
| 1 | Decitabine free | — | White Powder | 3 min Sonication | 8-10 |
| | base | | | | |
| 2 | Azacitidine free | — | White Powder | 1 sec. | 14 |
| | base | | | Mixing | |
| 3 | Decitabine | 1.04 | White Crystalline | 1 sec. | 280 (241)* |
| | HCl | | Powder | Mixing | |
| 4 | Decitabine | 1.00 | White Crystalline | 3 sec Sonication | 195 (137)* |
| | Mesylate | | Powder | | |
| 5 | Decitabine | 1.10 | White Powder | 5 min Sonication | 25-35 |
| | EDTA | | | | |
| 6 | Decitabine L- | 1.56 | White | 8 sec. | 25-35 |
| | Aspartate | | Crystalline | Sonication | |
| | | | Powder | | |
| 7 | Decitabine | 0.078 | White Crystalline | 5 sec. | 25-35 |
| | Maleate | | Powder | Sonication | |
| 8 | Decitabine L- | 1.58 | White | 10 sec. | 25-35 |
| | Glutamate | | Crystalline | Sonication | |
| | | | Powder | | |
| 9 | Decitabine | 0.99 | White Crystalline | 1 sec. | 80 (59)* |
| | Sulfite | | Powder | Mixing | |
| 10 | Decitabine | 1.06 | White | 5 sec. mixing | 50 (35)* |
| | Phosphate | | Powder | | |
| 11 | Decitabine | 0.091 | White | 5 sec. Sonication | 25-35 |
| | (+)-L-Tartrate | | Powder | | |
| 12 | Decitabine | 0.061 | White | 5 sec. | 25-35 |
| | Citrate | | Powder | Sonication | |
| 13 | Decitabine | 0.089 | Fine white | 3 sec. | 25-35 |
| | Lactate | | Crystalline | Mixing | |
| | | | Powder | | |
| 14 | Decitabine | 0.030 | White | 15 sec. | 25-35 |
| | Succinate | | Crystalline | Sonication | |
| | | | Powder | | |
| 15 | Decitabine | 0.17 | Fine white | 2 sec. | 25-35 |
| | Acetate | | crystalline | Sonication | |
| | | | Powder | | |
| 16 | Decitabine | 0.11 | White | 3 sec. | 25-35 |
| | Hexanoate | | Crystalline | Sonication | |
| | | | Powder | | |
| 17 | Decitabine | 0.15 | White | 4 sec. Sonication | 25-35 |
| | Butyrate | | Crystalline | | |
| | | | Powder | | |
| 18 | Decitabine | 0.19 | White | 2 sec. | 25-35 |
| | Propionate | | Crystalline | Sonication | |
| | | | Powder | | |
| 19 | Azacitidine | 1.02 | White Crystalline | 1 min Sonication | 205 (137)* |
| | Mesylate | | Powder | | |

^{#}Based on elemental analysis
*Decitabine or azacitidine free base equivalents

Table 3 shows the melting points and hydroscopicity of certain embodiments of the instant invention compared to free decitabine and free azacitidine. The observed melting (decomposition) points for decitabine hydrochloride (3) (130° C.) decitabine mesylate (4) (125° C.), for example, are different from that of decitabine free base crystalline anhydrate (1) (190° C.). The observed melting (decomposition) point for azacitidine mescylate (19) (138° C.) was also found to be different from that of azacitidine free base (2) (230° C.).

Table 3 also shows that certain salts are slightly more hydroscopic than the corresponding free base. Percent weight gained after one week in 56% relative humidity (RH) for decitabine hydrochloride (3) and decitabine mesylate (4) salts were similar to decitabine free base (1). At 98% RH, decitabine hydrochloride picked up considerably more moisture than decitabine—65.5% compared to only 2.88% weight gain. Decitabine mesylate, however, was determined to be no more hydroscopic than decitabine at 98% RH, showing only 2.84% weight gain. Nonetheless, azacitidine mesylate (19) was shown to be more hydroscopic than free azacitidine (2).

TABLE 3
Stability of decitabine and azacitidine salt forms in solid state

| | | | Melting Point | C₈H₁₂N₄O₄._{—} | Hygroscopicity-% | |
|---|---|---|---|---|---|---|
| | | pKₐ₁ of | (° C.) | Acid Molar | weight gain in 1 week | |
| Compound # | Sample | Acid Used | (Decompose) | Ratio | 56% RH | 98% RH |
| 1 | Decitabine | — | 190 | — | 0.68 | 2.88 |
| | free base | | | | | |
| 2 | Azacitidine | — | 230 | — | 1.74 | 5.61 |
| | free base | | | | | |
| 3 | Decitabine | −9 | 130 | 1.04 | 0.81 | 65.6 |
| | HCl | | | | | |
| 4 | Decitabine | −1.2 | 125 | 1.00 | 0.50 | 2.84 |
| | Mesylate | | | | | |
| 5 | Decitabine | 1.7 | 230 | 1.10 | 1.23 | 3.76 |
| | EDTA | | | | | |
| 6 | Decitabine L- | 1.9 | 190 | 1.56 | 3.23 | 4.21 |
| | Aspartate | | | | | |
| 7 | Decitabine | 1.9 | 210 | 0.078 | 0.76 | 7.2 |
| | Maleate | | | | | |
| 8 | Decitabine L- | 2.2 | 180 | 1.58 | 2.0 | 3.95 |
| | Glutamate | | | | | |
| 9 | Decitabine | 1.9 | 220 | 0.99 | 0.29 | 1.46 |
| | Sulfite | | | | | |
| 10 | Decitabine | 2.0 | 118 | 1.06 | 0.48 | 5.51 |
| | Phosphate | | | | | |
| 11 | Decitabine | 3.0 | 202 | 0.091 | 4.12 | 7.71 |
| | (+)-L-Tartrate | | | | | |
| 12 | Decitabine | 3.1 | 202 | 0.061 | 5.20 | 7.03 |
| | Citrate | | | | | |
| 13 | Decitabine | 3.9 | 195 | 0.089 | 0.79 | 11.13 |
| | L-Lactate | | | | | |
| 14 | Decitabine | 4.2 | 210 | 0.030 | 5.56 | 8.25 |
| | Succinate | | | | | |
| 15 | Decitabine | 4.8 | 206 | 0.17 | 0.53 | 4.47 |
| | Acetate | | | | | |
| 16 | Decitabine | 4.8 | 205 | 0.11 | 0.0 | 2.10 |
| | Hexanoate | | | | | |
| 17 | Decitabine | 4.8 | 204 | 0.15 | 0.10 | 1.93 |
| | Butyrate | | | | | |
| 18 | Decitabine | 4.9 | 200 | 0.19 | 0.58 | 2.07 |
| | Propionate | | | | | |
| 19 | Azacitidine | −1.2 | 138 | 1.02 | 6.05 | 38.11 |
| | Mesylate | | | | | |

Table 4 depicts the aqueous stability of certain decitabine and azacitidine salts of the present invention. Aqueous stability was determined in phosphate buffer at pH 7.0 and pH 2.5 at a drug concentration of 0.5 mg/mL. The assay conditions were: mobile phase-mixture of 40±0.5 mL of methanol and 2000 mL of 10 mM ammonium acetate; column temperature of 15±2° C.; auto sampler temperature of 5° C.; flow rate of 1.7 mL/min; injection volume of 5 μL; detector wavelength of 220 nm; and analysis time of 25 minutes.

The solution stability of some of the decitabine salts in 0.05 M phosphate buffer solution at pH of 7.0 and 2.5 are at least as stable as decitabine free base. At pH of 7.0, decitabine hydrochloride (3) and decitabine free base (1) gave similar percent recoveries after approximately 30 minutes (87.59% and 87.17%) and 24 hours (81.07% and 84.07%, respectively) at ambient condition. Decitabine mesylate (4) exhibited slightly better percent recovery after 30 minutes and 24 hours (91.19% and 89.49%, respectively) at pH 7.0.

At pH of 2.5, decitabine mesylate (4) and decitabine free base (1) exhibited similar percent recovery after approximately 30 minutes (55.96% and 57.09%) and 24 hours (48.77% and 50.38%, respectively) at ambient condition. Decitabine hydrochloride (3) gave considerably better percent recovery after 30 minutes (77.89%), but eventually decreased to a value (49.90%) similar to decitabine free base (1). Decitabine L-aspartate (6) and decitabine sulfite (9) also appear to stabilize decitabine rather well. For example, the stability of decitabine sulfite (9) is improved at pH of 2.5 (95.96% after 30 minutes and 92.96% after 24 hours) compared with decitabine free base (1) (57.09% after 30 minutes and 50.8% after 24 hours).

With respect to azacitidine mesylate (19), the stability of this 1:1 salt is slightly less than the free azacitidine base (2).

TABLE 4
Stability of salts in 0.05 M phosphate buffer solution (0.5 mg/mL) at pH 7.0 and 2.5.

| | | | C₈H₁₂N₄O₄._{—} | Potency Found (%) | | Potency Found (%) | |
|---|---|---|---|---|---|---|---|
| | | pKₐ₁ of | Acid Molar | At pH 7.0 | | At pH 2.5 | |
| Compound # | Sample | Acid Used | Ratio | t = 0.5 hr | t = 24 hr | t = 0.5 hr | t = 24 hr |
| 1 | Decitabine free | — | — | 87.17 | 84.07 | 57.09 | 50.38 |
| | base | | | | | | |
| 2 | Azacitidine free | — | — | 86.74 | 79.43 | 73.62 | 54.85 |
| | base | | | | | | |
| 3 | Decitabine | −9 | 1.04 | 87.59 | 81.07 | 77.89 | 49.90 |
| | HCl | | | | | | |
| 4 | Decitabine | −1.2 | 1.00 | 91.19 | 89.49 | 55.96 | 48.77 |
| | Mesylate | | | | | | |
| 5 | Decitabine | 1.7 | 1.10 | 66.05 | 56.63 | 31.14 | 27.18 |
| | EDTA | | | | | | |
| 6 | Decitabine L- | 1.9 | 1.56 | 97.37 | 87.44 | 71.79 | 63.77 |
| | Aspartate | | | | | | |
| 7 | Decitabine | 1.9 | 0.078 | 87.56 | 80.54 | 52.14 | 46.54 |
| | Maleate | | | | | | |
| 8 | Decitabine L- | 2.2 | 1.58 | 89.10 | 78.46 | 60.82 | 51.62 |
| | Glutamate | | | | | | |
| 9 | Decitabine | 1.9 | 0.99 | 94.90 | 83.78 | 95.96 | 92.96 |
| | Sulfite | | | | | | |
| 10 | Decitabine | 2.0 | 1.06 | 85.97 | 79.78 | 80.31 | 42.42 |
| | Phosphate | | | | | | |
| 11 | Decitabine | 3.0 | 0.091 | 96.31 | 92.53 | 57.10 | 50.96 |
| | (+)-L-Tartrate | | | | | | |
| 12 | Decitabine | 3.1 | 0.061 | 92.01 | 88.35 | 57.50 | 50.64 |
| | Citrate | | | | | | |
| 13 | Decitabine | 3.9 | 0.089 | 88.38 | 88.03 | 62.81 | 55.27 |
| | L-Lactate | | | | | | |
| 14 | Decitabine | 4.2 | 0.030 | 87.35 | 80.58 | 62.81 | 54.89 |
| | Succinate | | | | | | |
| 15 | Decitabine | 4.8 | 0.17 | 89.73 | 84.06 | 56.39 | 50.31 |
| | Acetate | | | | | | |
| 16 | Decitabine | 4.8 | 0.11 | 93.77 | 88.24 | 59.40 | 52.84 |
| | Hexanoate | | | | | | |
| 17 | Decitabine | 4.8 | 0.15 | 94.63 | 88.25 | 58.59 | 50.70 |
| | Butyrate | | | | | | |
| 18 | Decitabine | 4.9 | 0.19 | 94.63 | 88.89 | 62.36 | 56.60 |
| | Propionate | | | | | | |
| 19 | Azacitidine | −1.2 | 1.02 | 77.47 | 65.79 | 64.56 | 49.94 |
| | Mesylate | | | | | | |

3. Thermal Analyses of Decitabine and Azacitidine Salts

For some of the salt forms, “fingerprint” analyses that include Differential Scanning Calorimetry (DSC), Thermo Gravimetric Analysis (TGA), X-ray Diffraction (XRD) and Infrared (IR) Spectroscopic analysis are provided herein. Numerical values for DSC provided herein are intended to be each modified by “about.” For example, DSC values provided herein represent the given numerical value ±1° C., ±2° C., ±3° C., ±4° C., ±5° C., ±6° C., ±7° C., ±8° C., ±9° C., ±10° C. and±at least 10° C.

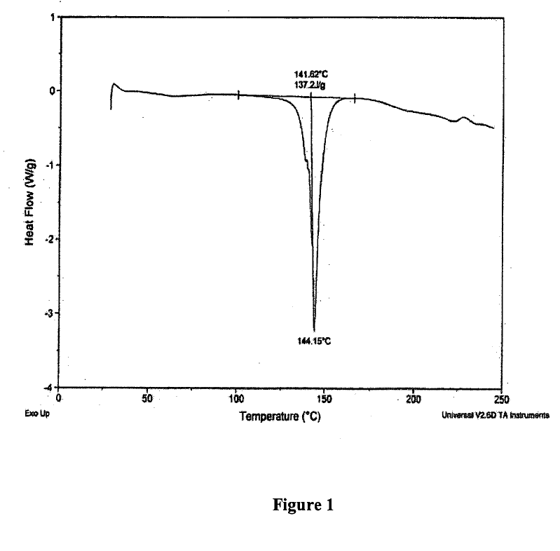

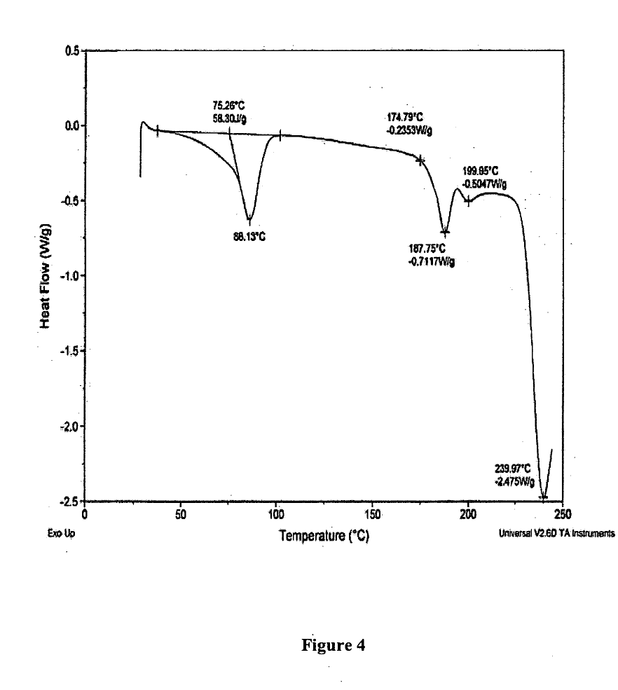

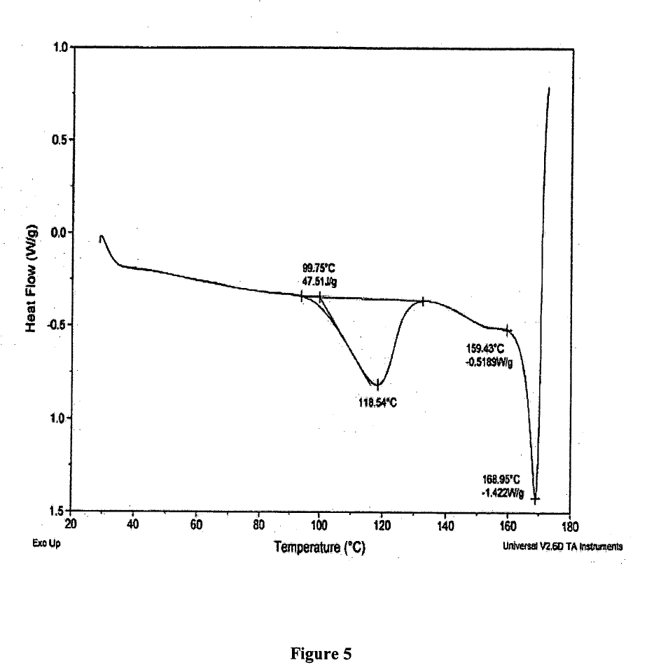

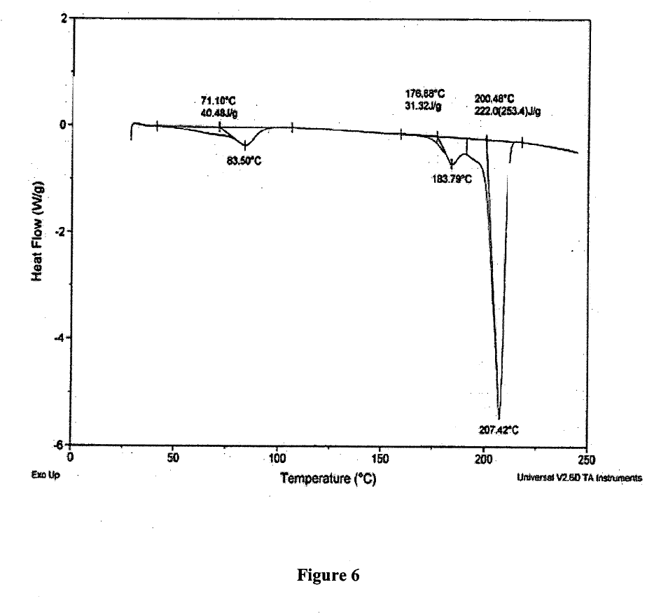

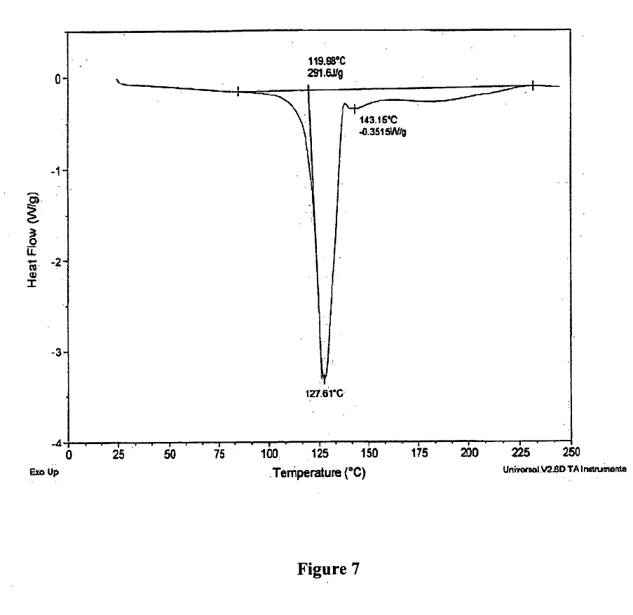

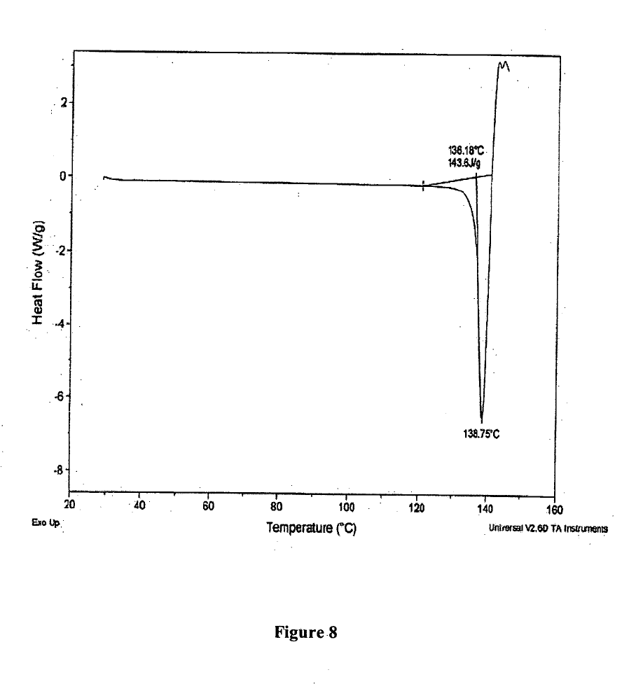

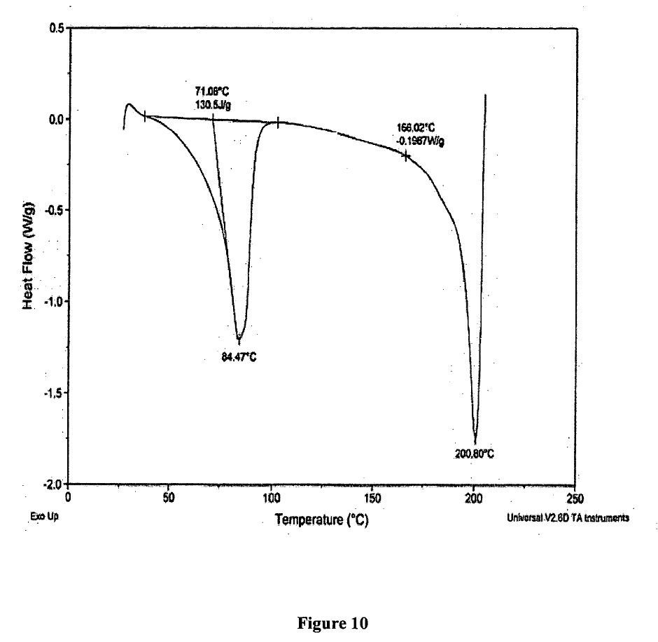

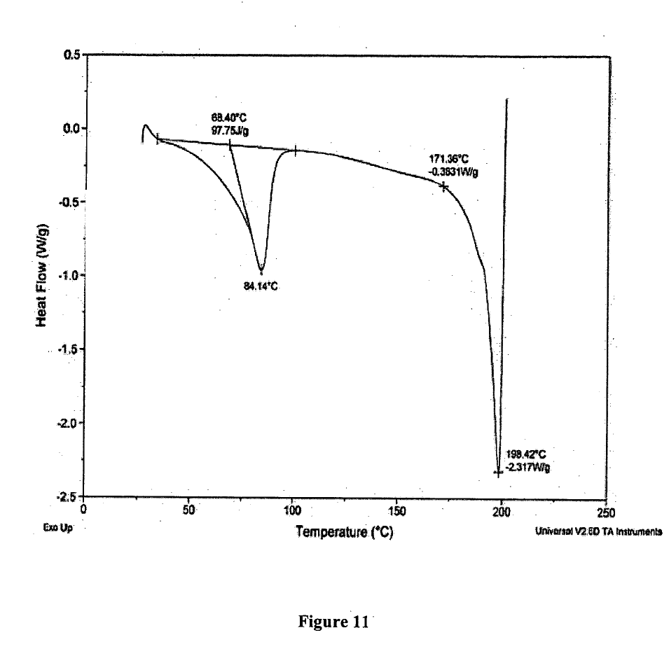

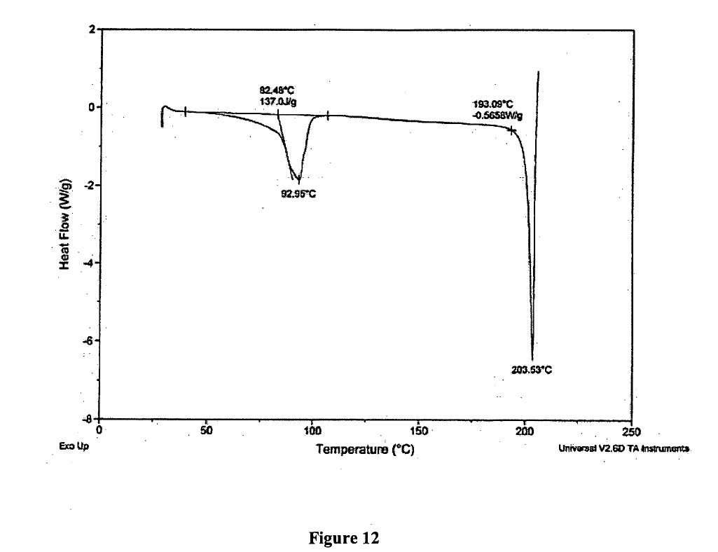

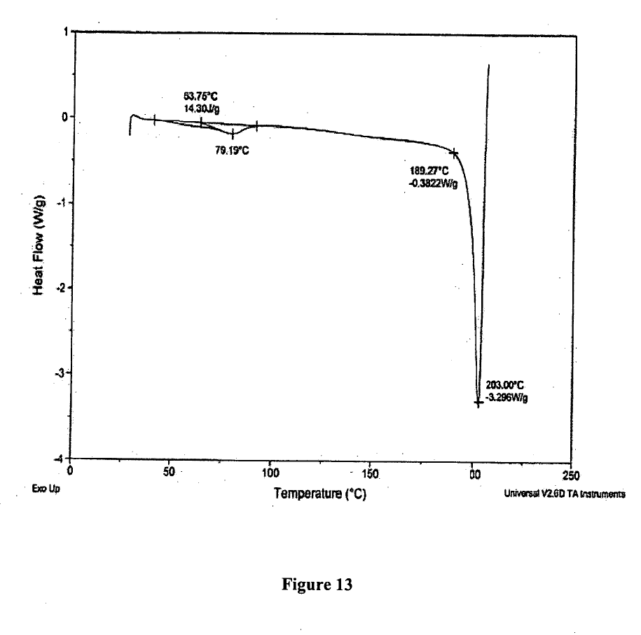

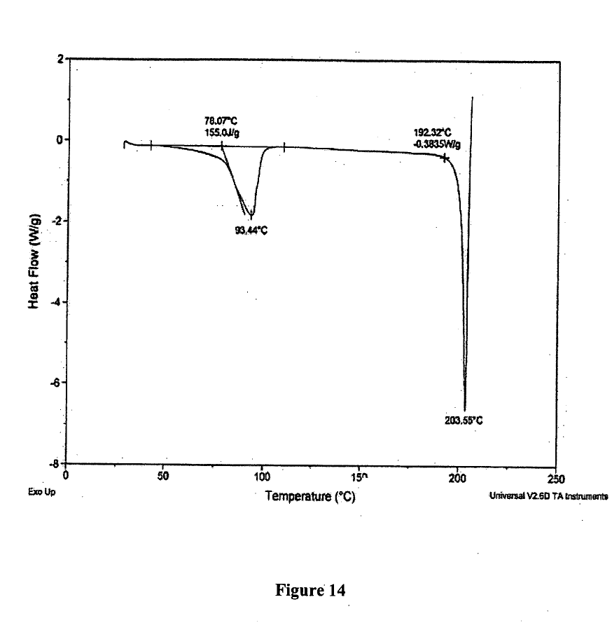

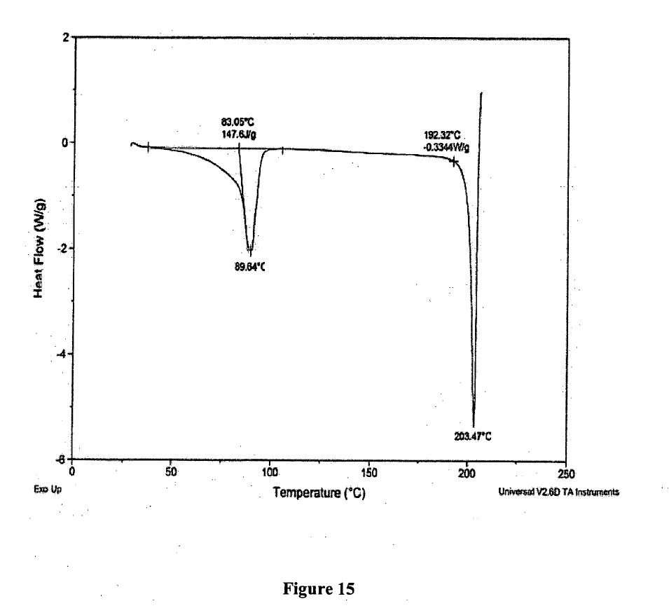

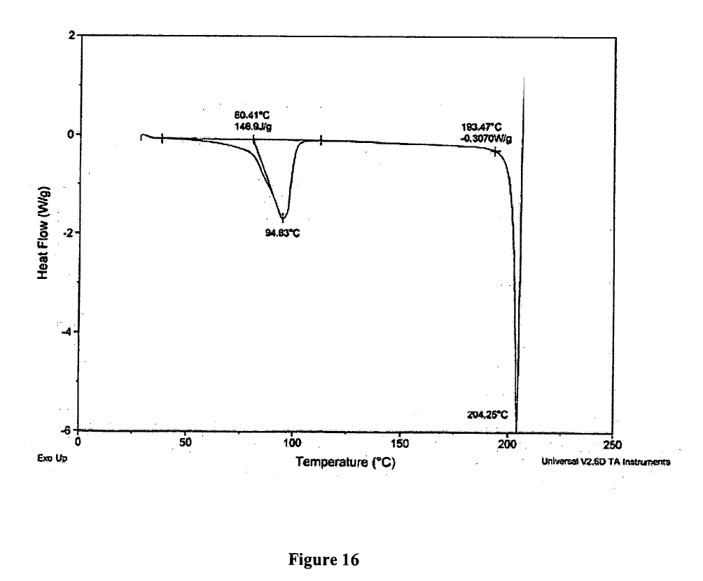

As mentioned above, the observed melting (decomposition) points shown in Table 3 for decitabine hydrochloride (3) (130° C.) and decitabine mesylate (4) (125° C.) are different from that of decitabine free base crystalline anhydrate (1) (190° C.). These values were corroborated by differential scanning calorimetry (DSC) plots (at 10° C. per minute, ambient to 250° C.). FIGS. 1-17 illustrate DSC plots of decitabine hydrochloride (3), decitabine mesylate (4), decitabine EDTA (5), decitabine L-aspartate (6), decitabine maleate (7), decitabine L-glutamate (8), decitabine sulfite (9), decitabine phosphate (10), decitabine tartrate (11), decitabine citrate (12), decitabine L-(+)-lactate (13), decitabine succinate (14), decitabine acetate (15), decitabine hexanoate (16), decitabine butyrate (17), decitabine propionate (18), and azacitidine mesylate (19), respectively.

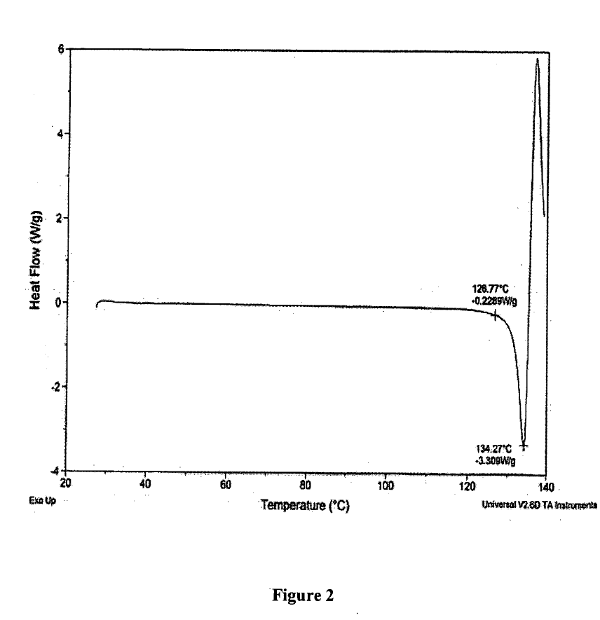

As FIG. 1 illustrates, decitabine hydrochloride (3) undergoes a major thermal event starting around 130° C. and culminating at 144° C. As illustrated in FIG. 2, decitabine mesylate (4) has a major thermal event starting around 125° C. and culminating at 134° C. These DSC endothermic events with an onset near 125-130° C. correspond which is accompanied by an exothermic event. This behavior indicates that both decitabine hydrochloride and decitabine mesylate melt with decomposition.

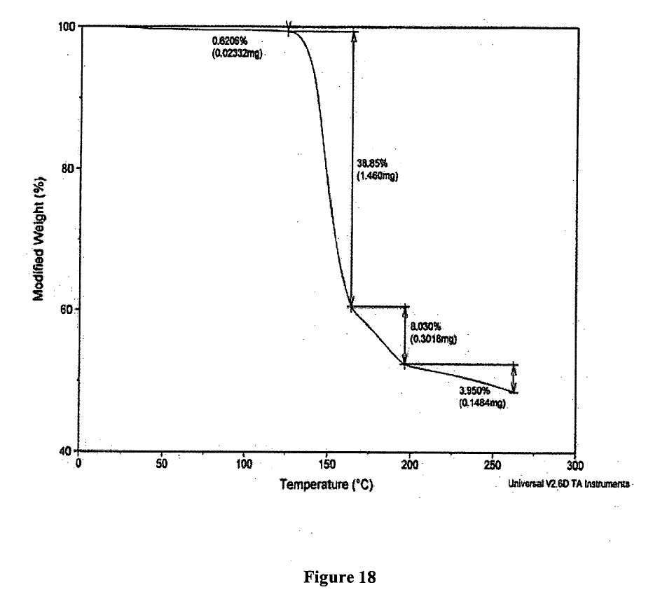

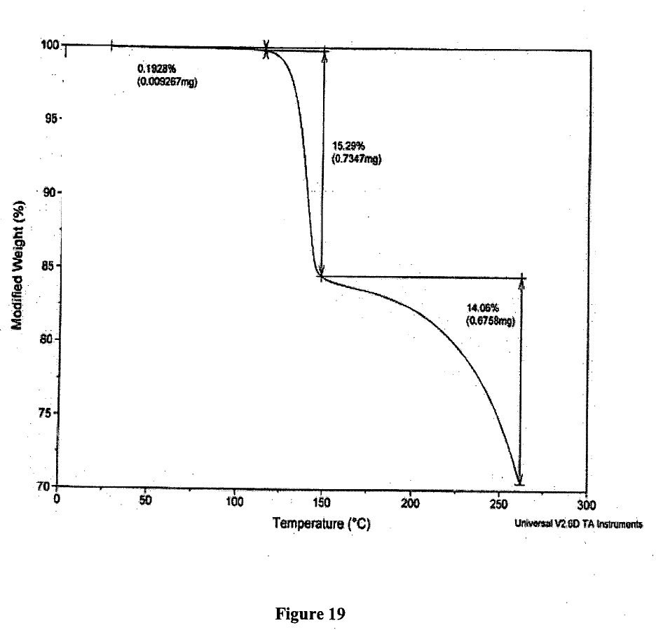

Thermal analyses of these two novel salts suggest that they are anhydrate form. FIGS. 18 and 19 illustrate TGA plots of decitabine hydrochloride (3) and decitabine mesylate (4), respectively. TGA plot for each does not show a weight loss up to the decomposition point of the sample. As FIG. 18 illustrates, the TGA plot of decitabine hydrochloride (3) shows a steep decomposition curve appearing around 150° C. and accounting for over 38% weight loss. The decomposition curve finally plateaus around 200 to 250° C. Without being limited to a particular hypothesis, it appears that loss of hydrogen chloride during decomposition is accompanied by cleavage of the triazine ring around 150° C., as depicted below.

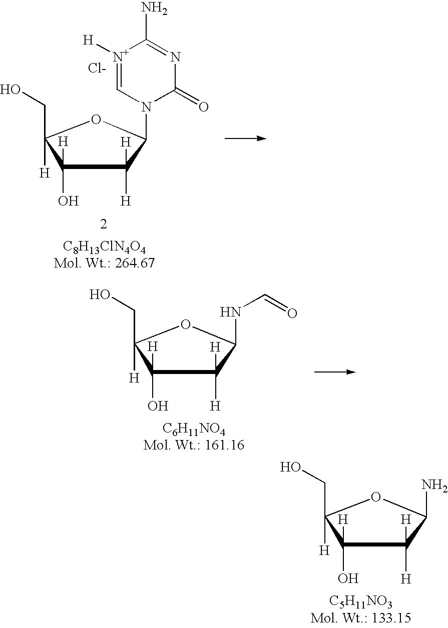

FIG. 19 illustrates the TGA plot of decitabine mesylate (4), where two major consecutive decomposition events appear around 150° C. and around 200 to 250° C. The first event accounts for 15% weight lost, while the second accounts for 14%. While not being limited to a particular hypothesis, decitabine mesylate may decompose in stages similar to those of decitabine hydrochloride, as depicted below. For example, decitabine mesylate decomposition may be accompanied by cleavage of the triazine ring, as hypothesized in the case of decitabine hydrochloride. In contrast, however, cleavage of the triazine in free decitabine does not occur until around 190° C.

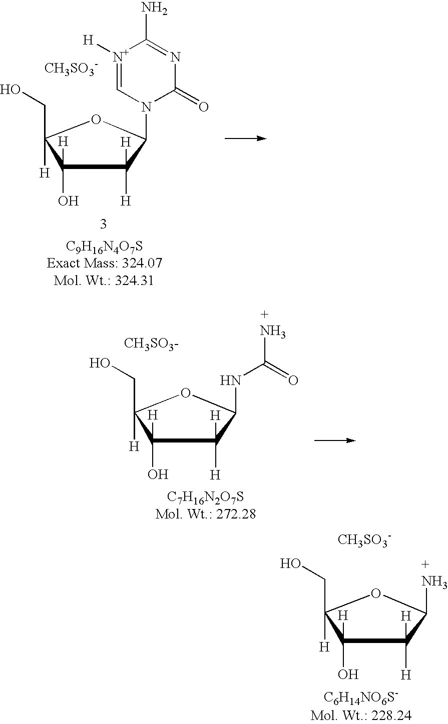

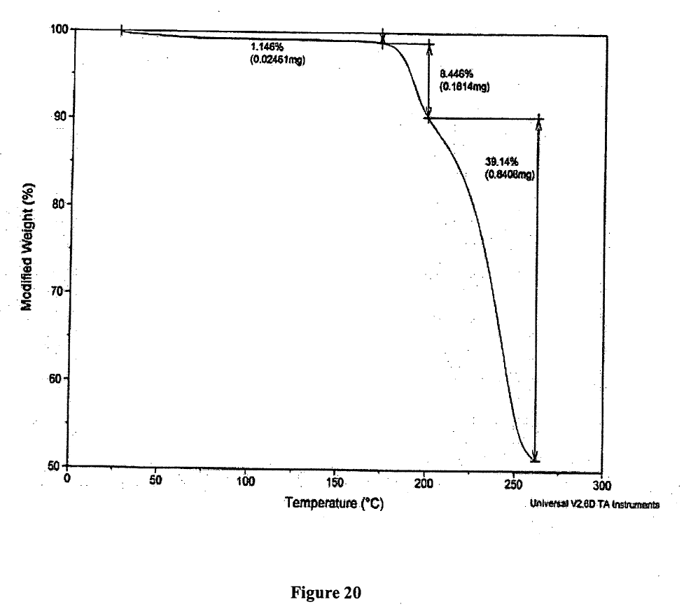

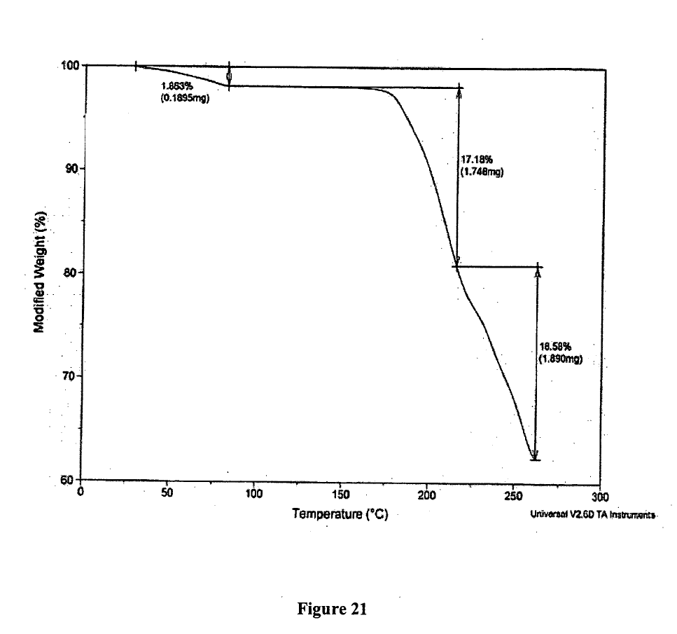

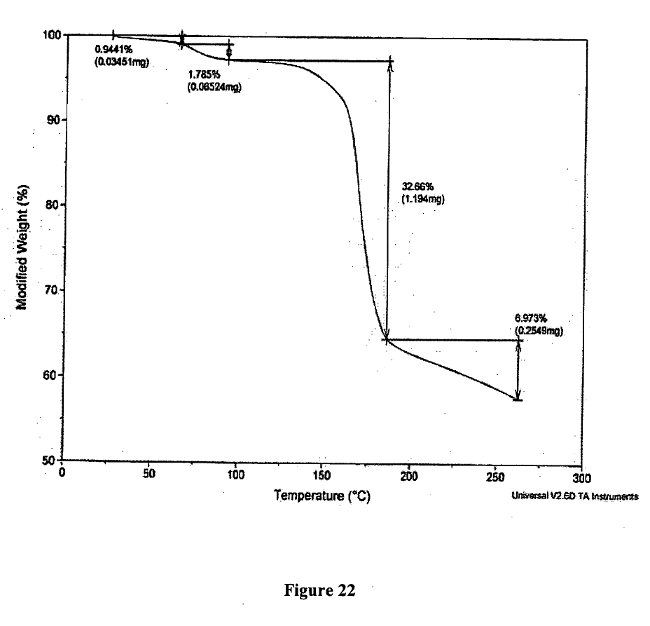

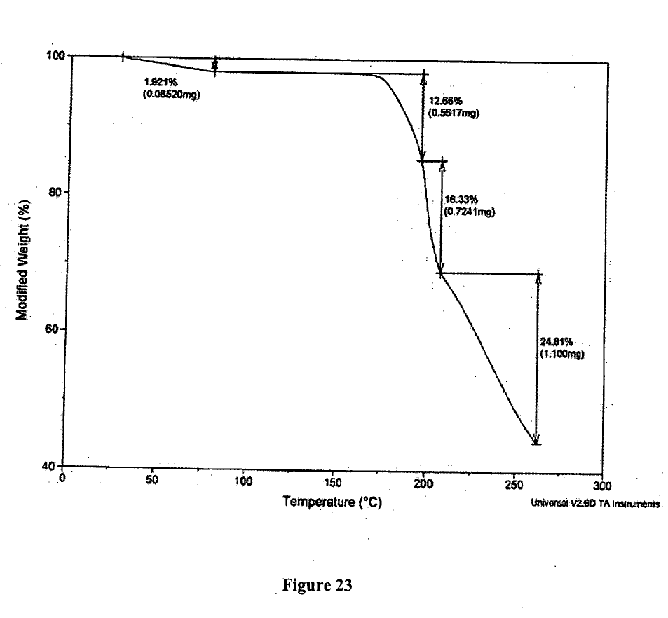

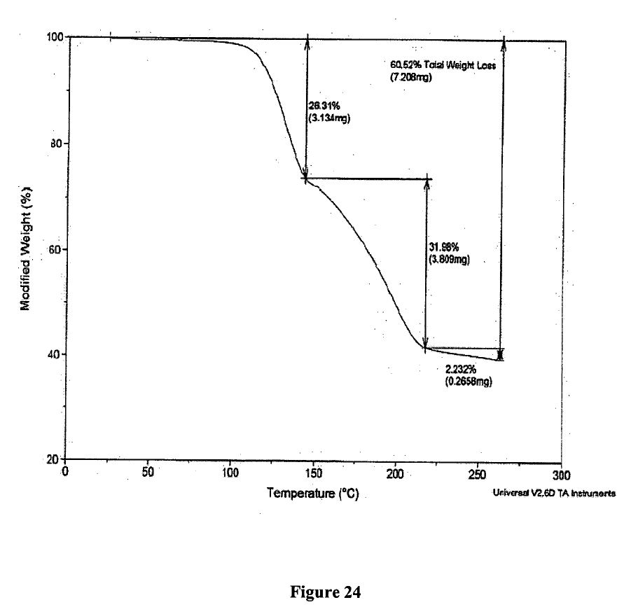

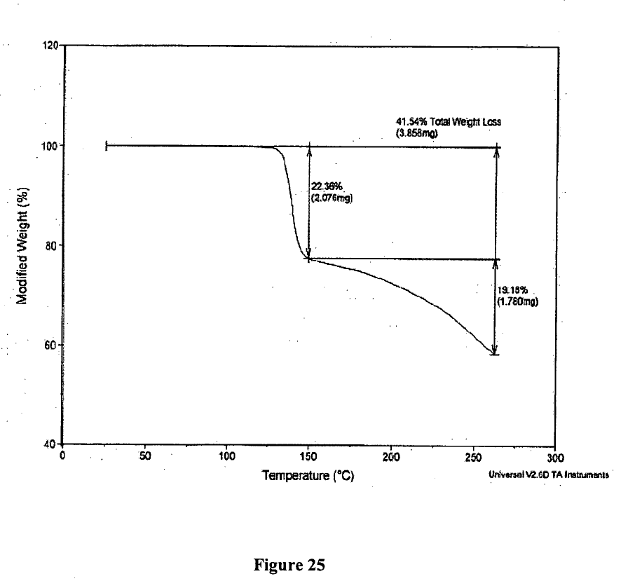

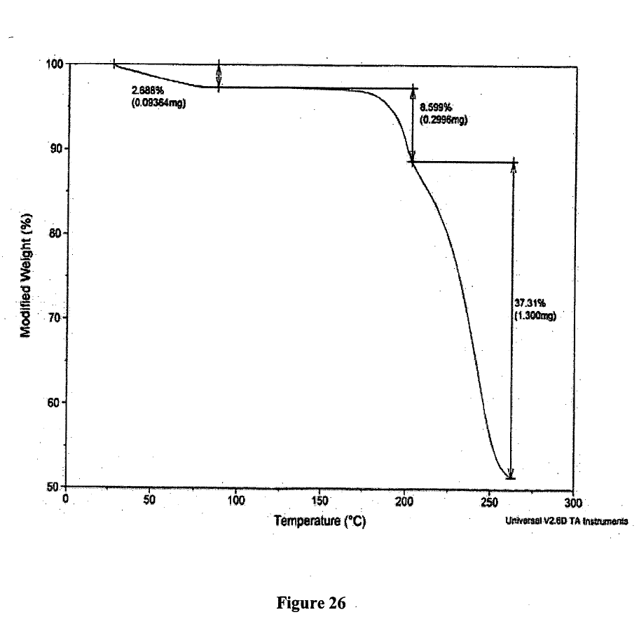

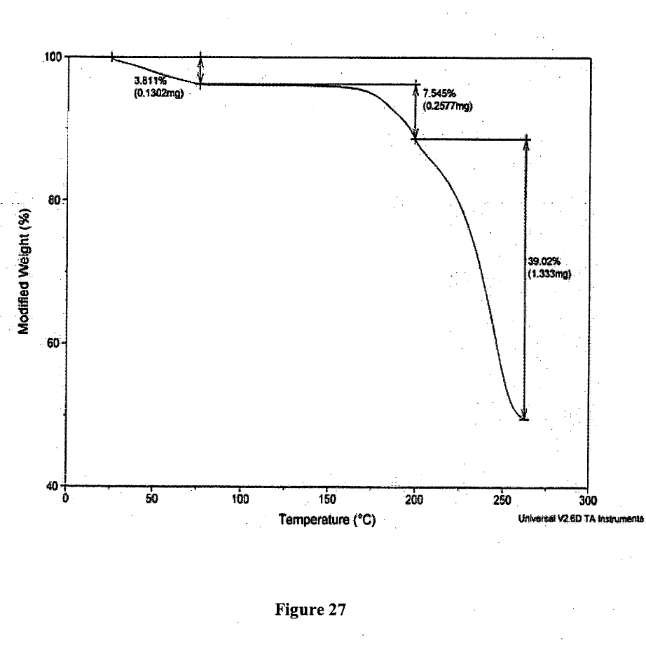

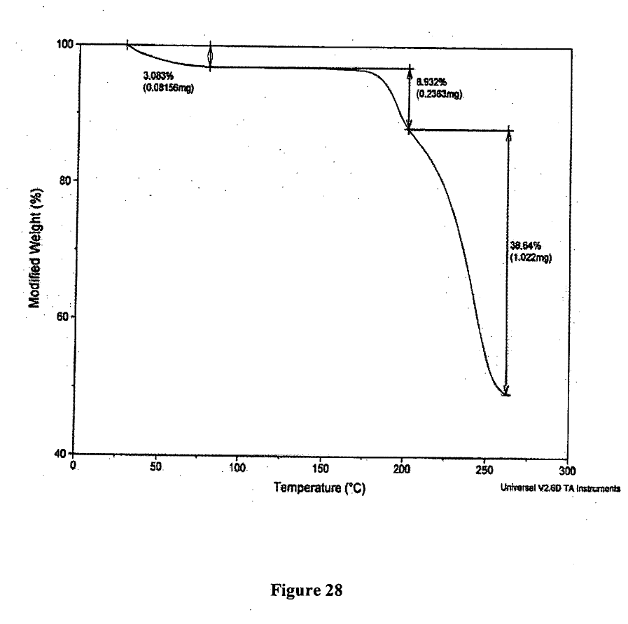

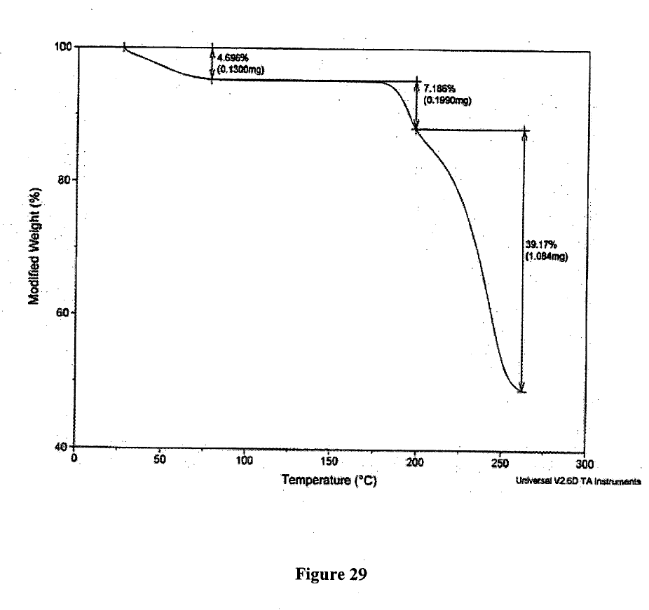

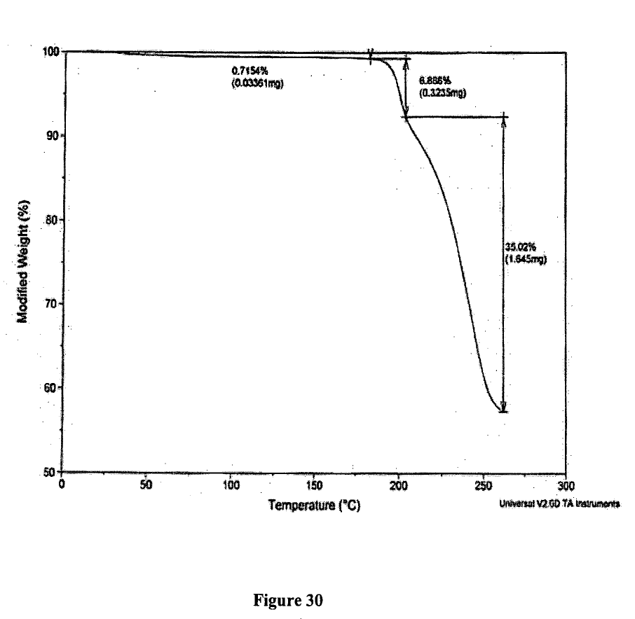

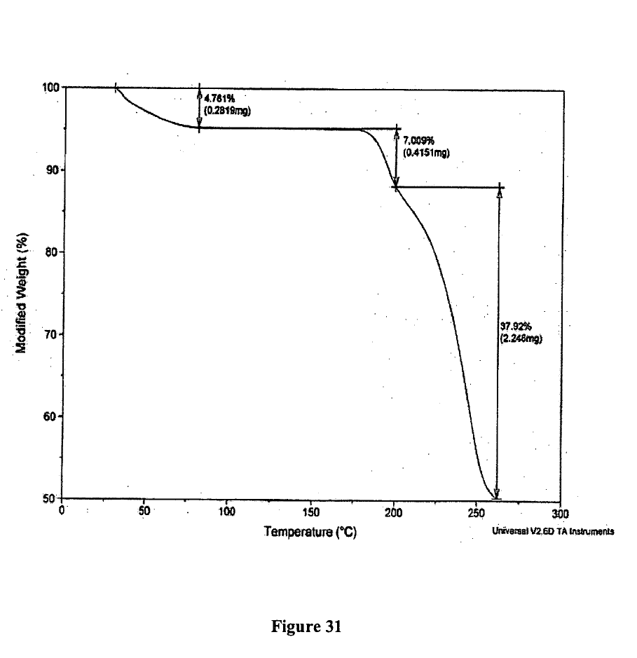

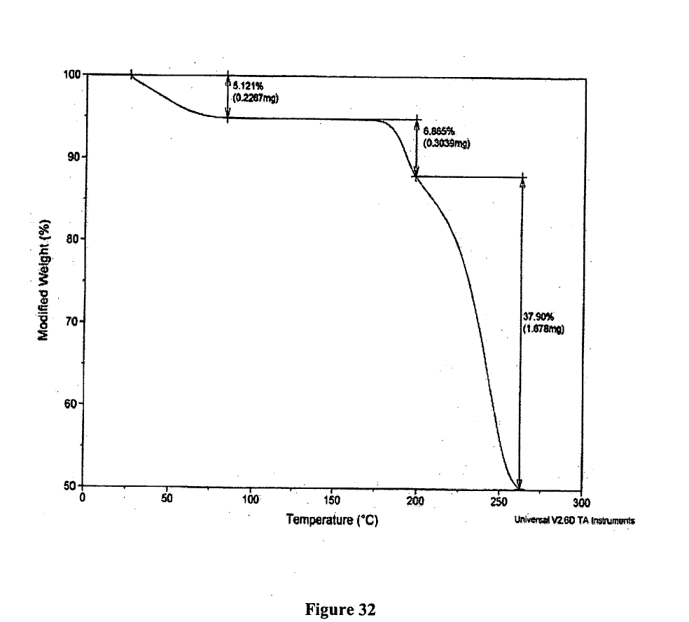

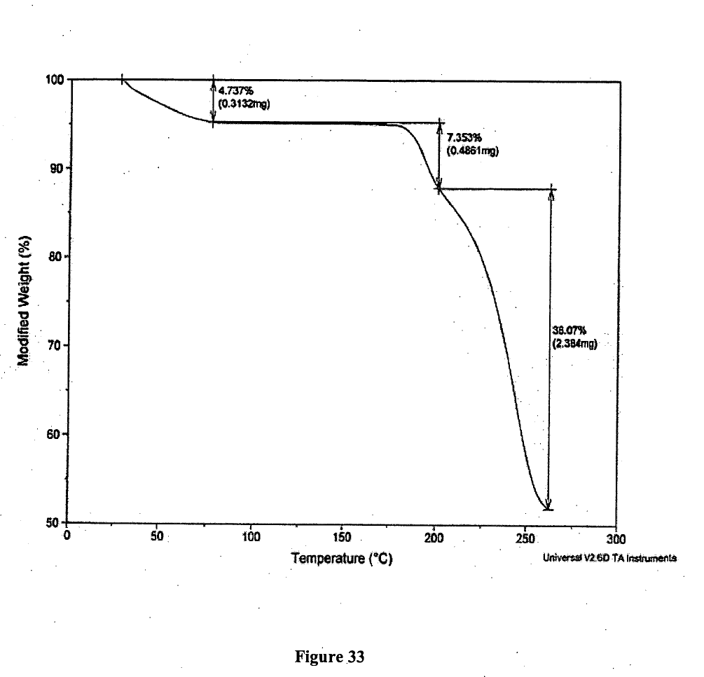

FIGS. 20-34 illustrate TGA plots for additional salts of the instant invention, namely decitabine EDTA (5), decitabine L-aspartate (6), decitabine maleate (7), decitabine L-glutamate (8), decitabine sulfite (9), decitabine phosphate (10), decitabine tartrate (11), decitabine citrate (12), decitabine L-(+)-lactate (13), decitabine succinate (14), decitabine acetate (15), decitabine hexanoate (16), decitabine butyrate (17), decitabine propionate (18), and azacitidine mesylate (19), respectively.

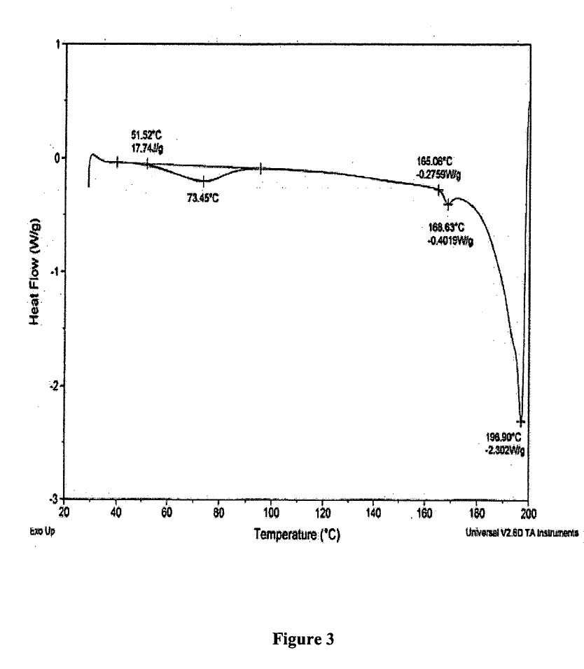

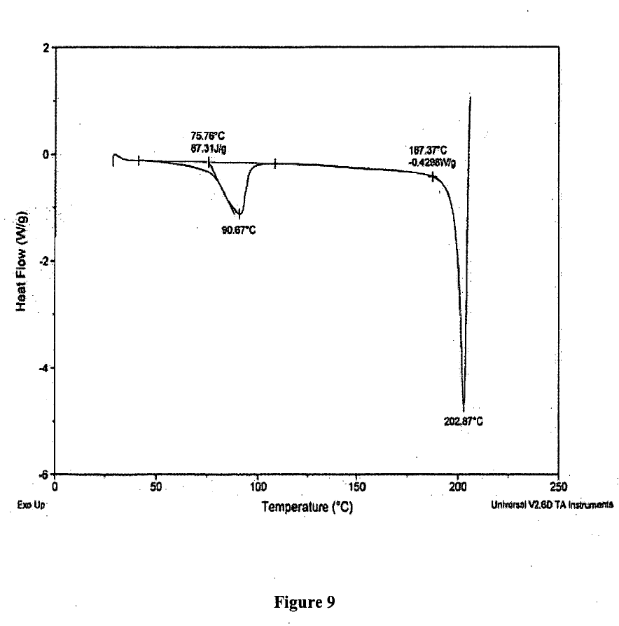

From the DSC and TGA plots for decitabine EDTA (5), decitabine L-aspartate (6), decitabine maleate (7), decitabine L-glutamate (8), decitabine sulfite (9), and decitabine phosphate (10) (FIGS. 3-8 and 20-25, respectively), it can be seen that these salts are not free decitabine. Accordingly, decitabine sulfite (9) and decitabine phosphate (10) have solubility of 80 mg/mL and 50 mg/mL, respectively or equivalent to 59 mg/mL and 35 mg/mL of free base, respectively (as shown in Table 2 above). From the DSC and TGA plots for decitabine tartrate (11), decitabine citrate (12), decitabine L-(+)-lactate (13), decitabine succinate (14), decitabine acetate (15), decitabine hexanoate (16), decitabine butyrate (17), decitabine propionate (18) (FIGS. 9-16 and 26-33, respectivley), it can be seen that these crude salt mixtures predominantly contain decitabine. As such, solubility measurement of these crude salt mixtures shown in Table 2 may not be representative of pure 1:1 molar equivalent salts. Nonetheless, as shown in Table 4, the stabilities of these crude salt mixtures are at least as good as decitabine, if not slightly better.

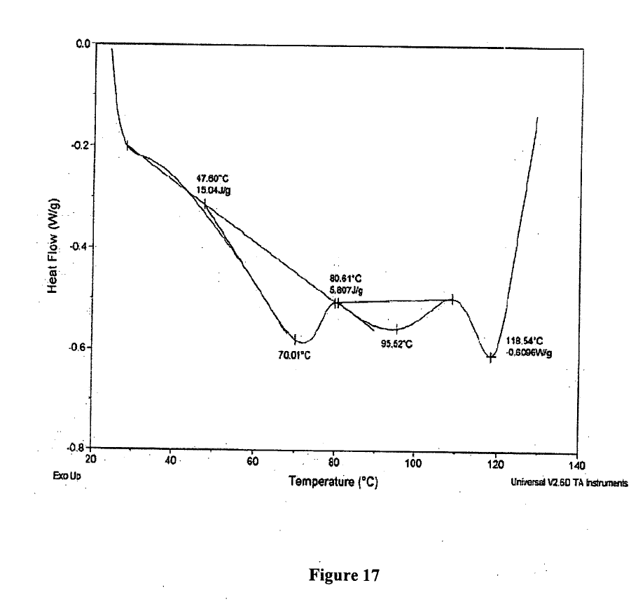

As mentioned above, the observed melting (decomposition) point shown in Table 3 for azacitidine mesylate (19) (138° C.) is different from that of azacitidine free base (2) (230° C.). This value was corroborated by DSC plot (at 10° C. per minute, ambient to 250° C.), illustrated in FIG. 17. As FIG. 17 shows, azacitidine mesylate (19) undergoes major thermal events around 70, 95 and 118° C. These endothermic events with an onset near 70-130 ° C. correspond to the melt, which is accompanied exothermic event. This behavior indicates that azacitidine mesylate can melt with decomposition.

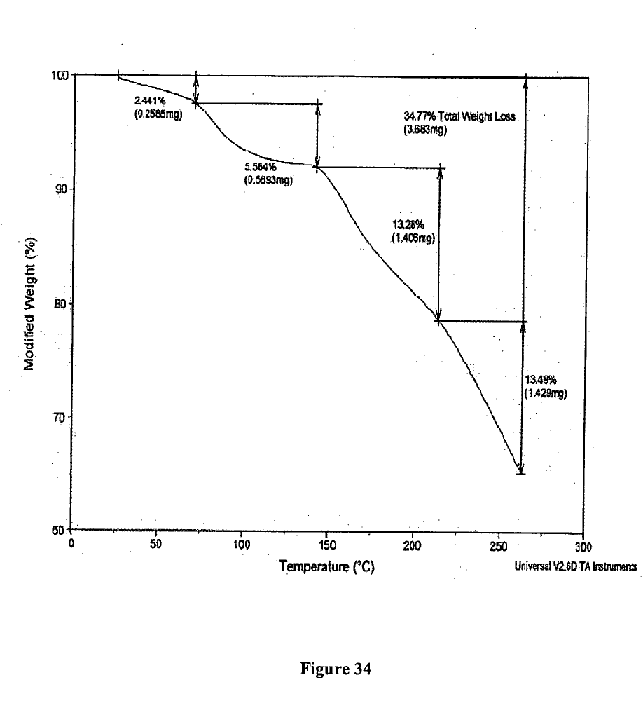

Further, as illustrated in FIG. 34, the TGA plot of azacitidine mesylate, a series of major decomposition events appear around 70° C. to 250° C. The decomposition events prior to 150° C. accounts for less than 10% weight lost, while consecutive decomposition up to 250° C. accounts for almost 50% weight lost.

4. X-Ray Diffraction and Infra-Red Spectra for Decitabine and Azacitidine Salts

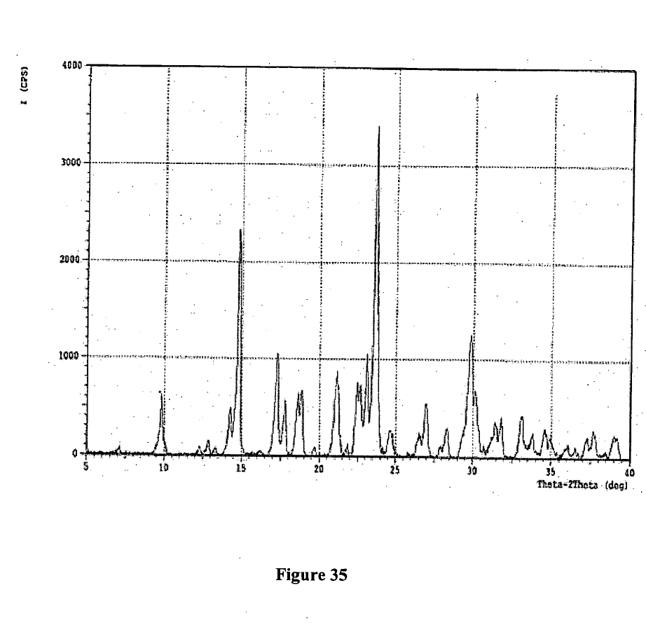

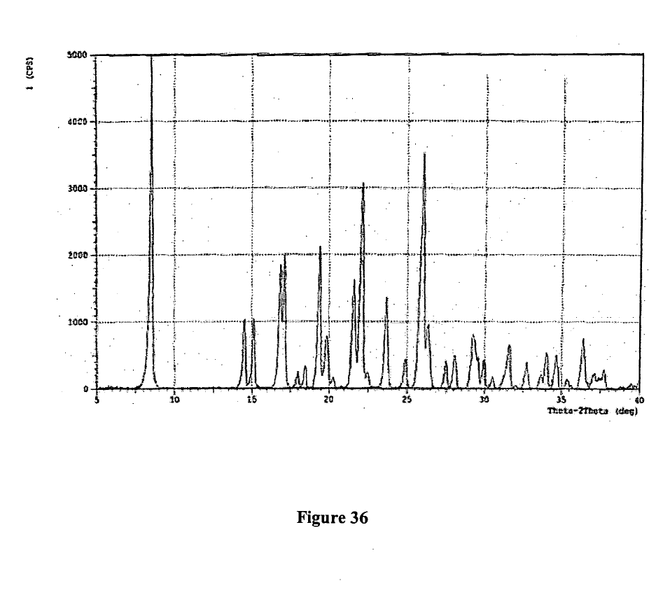

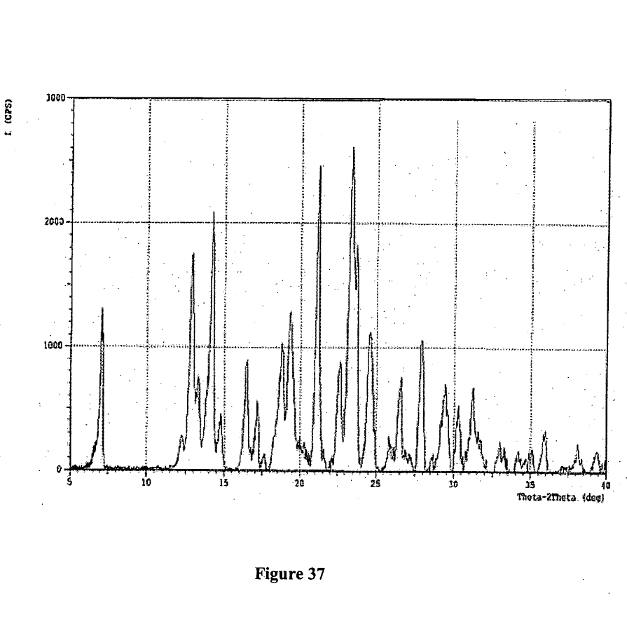

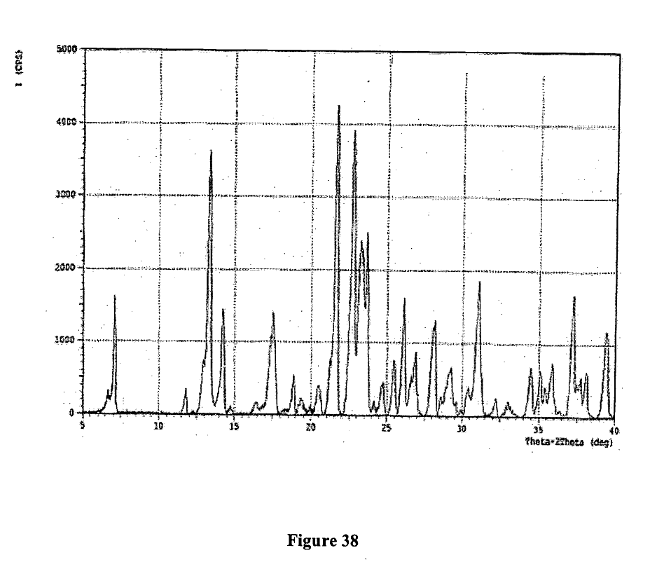

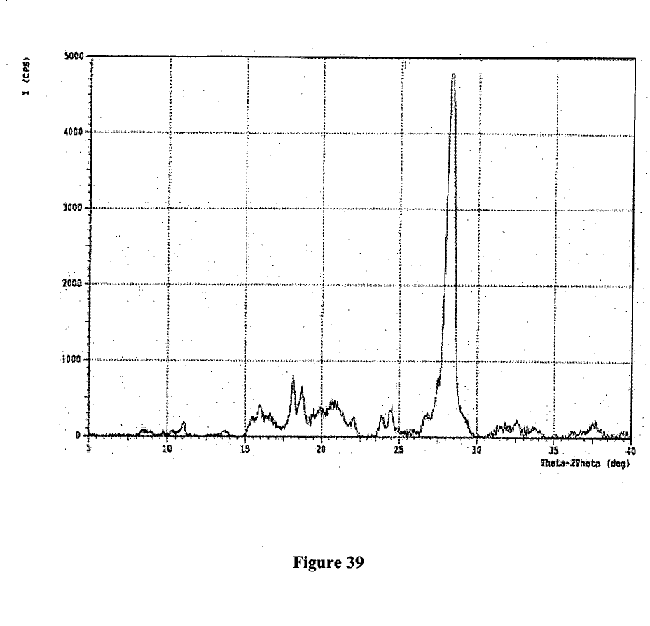

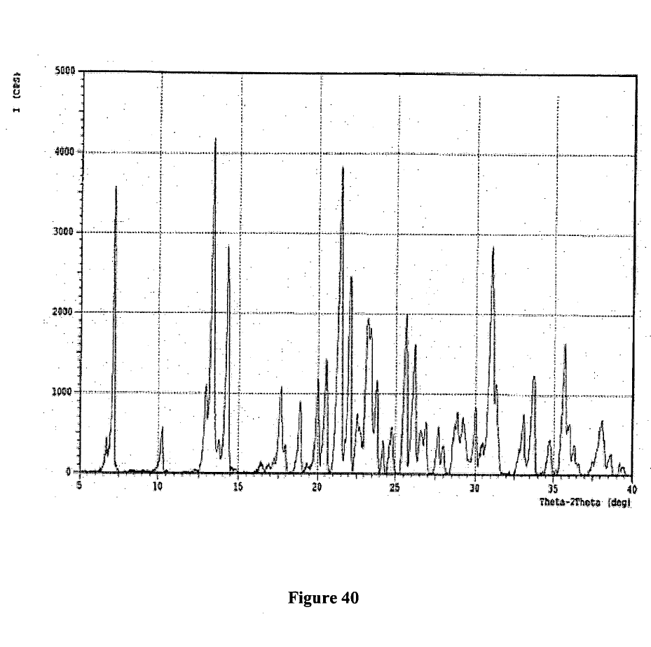

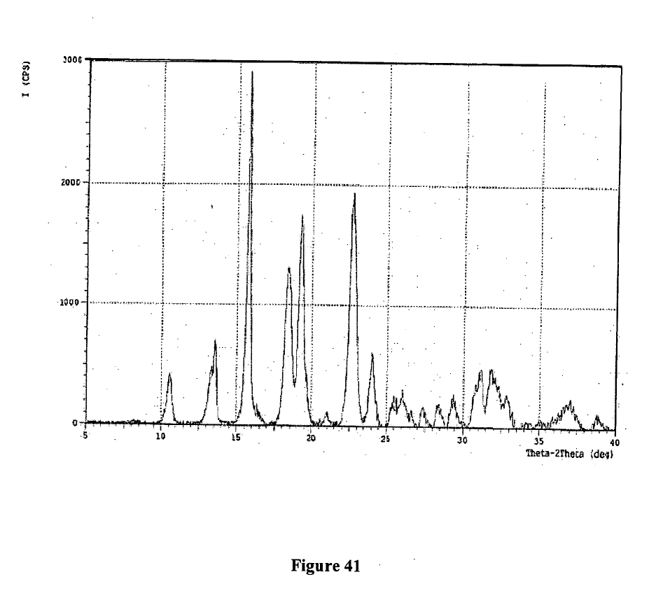

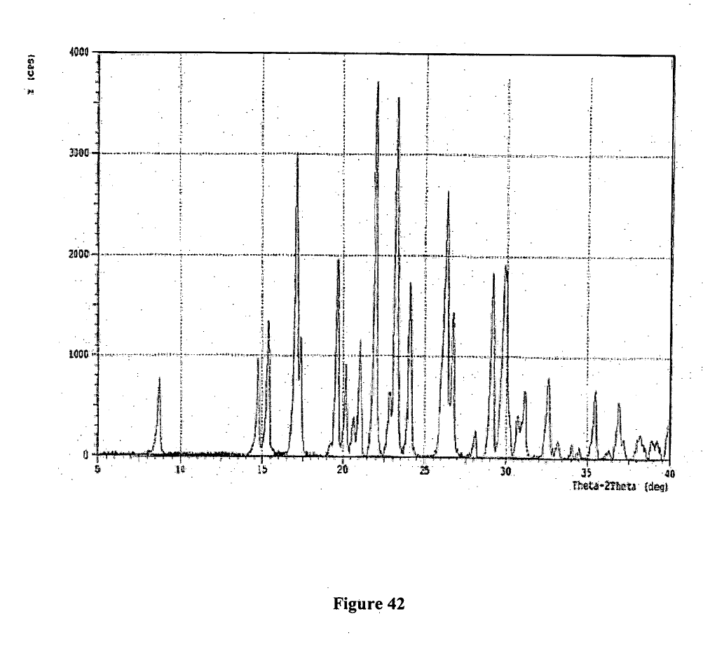

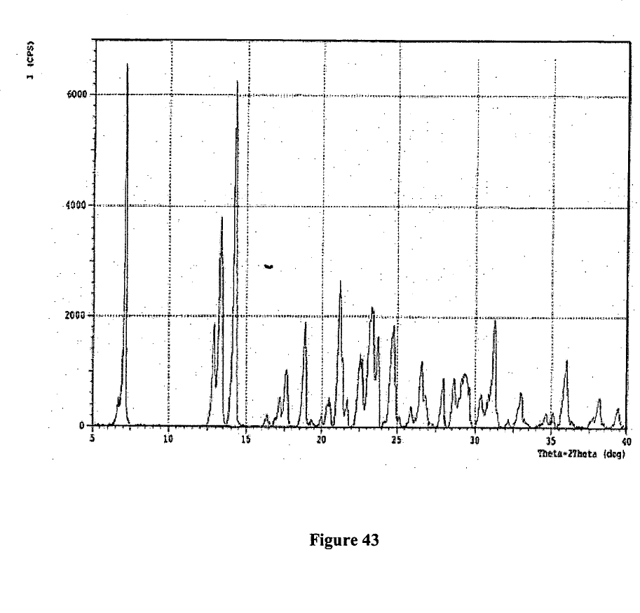

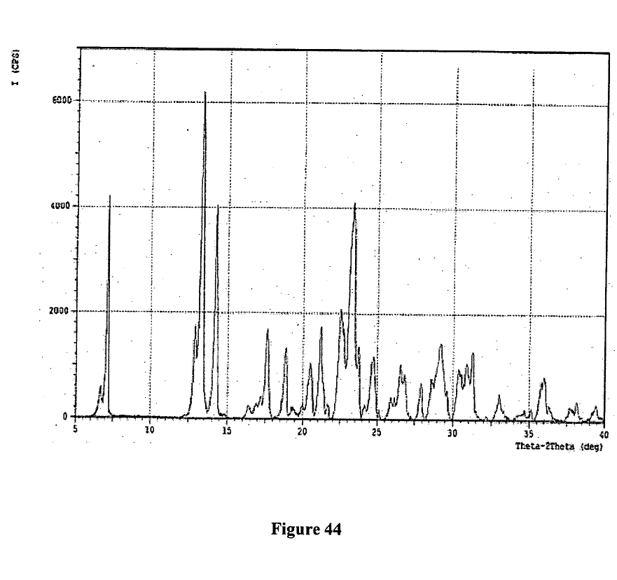

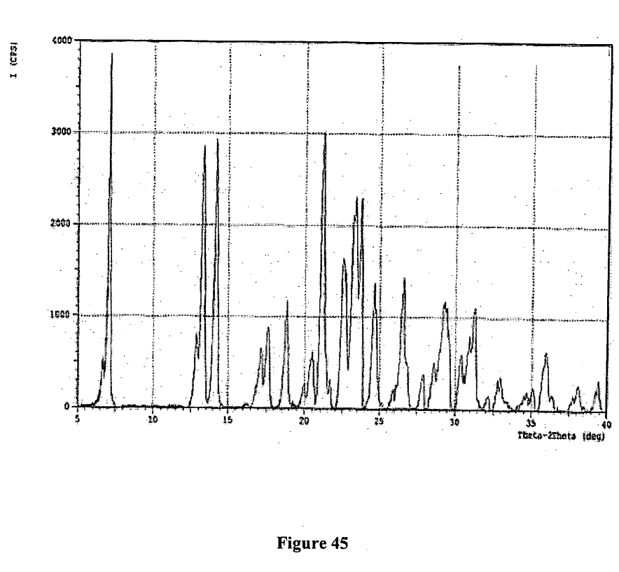

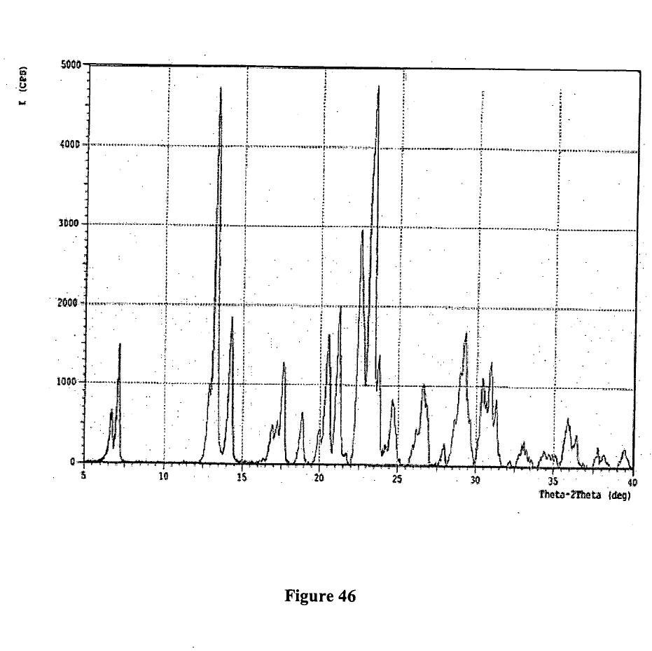

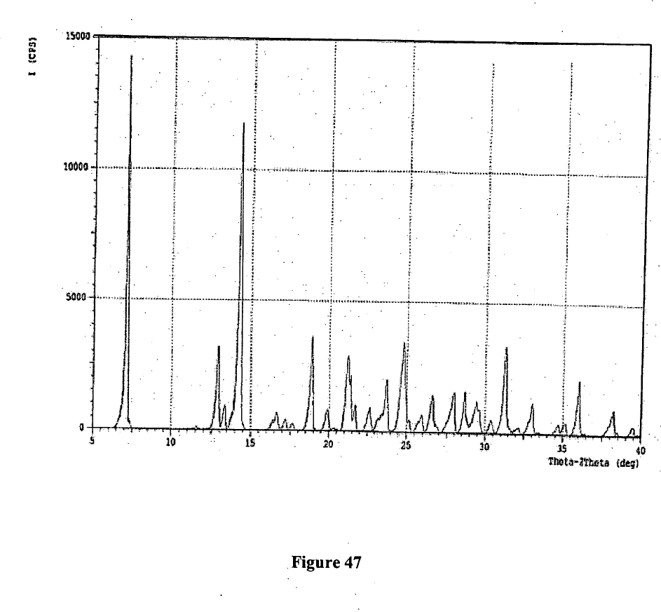

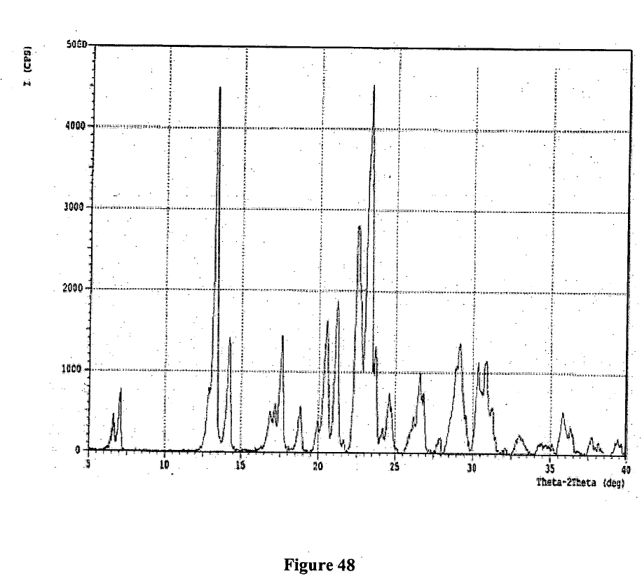

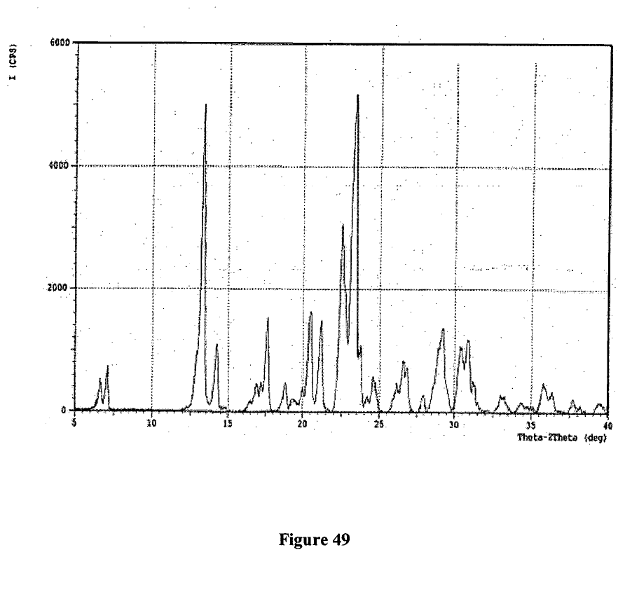

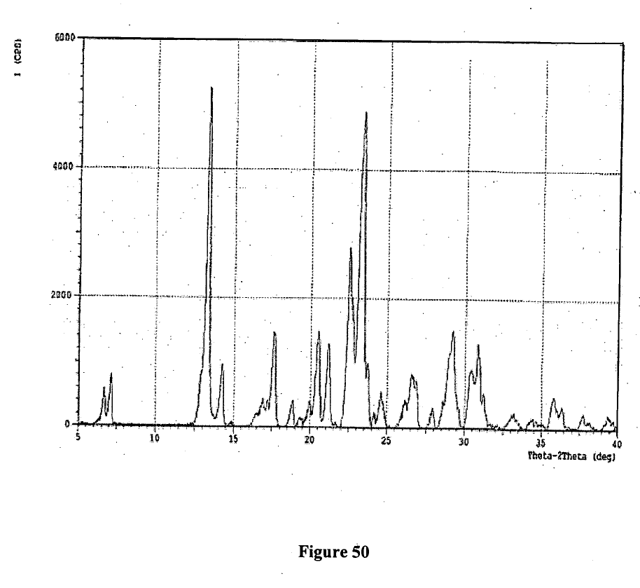

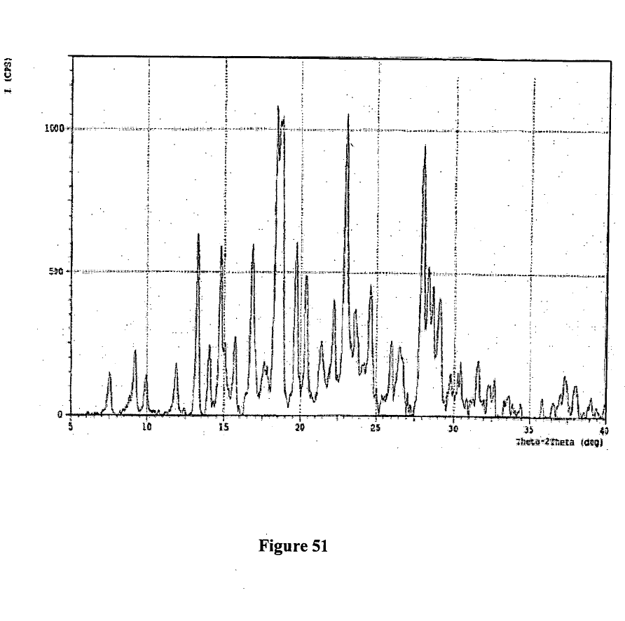

Fingerprint XRD also were obtained for certain embodiments of the instant invention. FIGS. 35-51 illustrate XRD patterns of decitabine hydrochloride (3), decitabine mesylate (4), decitabine EDTA (5), decitabine L-aspartate (6), decitabine maleate (7), decitabine L-glutamate (8), decitabine sulfite (9), decitabine phosphate (10), decitabine tartrate (11), decitabine citrate (12), decitabine L-(+)-lactate (13), decitabine succinate (14), decitabine acetate (15), decitabine hexanoate (16), decitabine butyrate (17), decitabine propionate (18), and azacitidine mesylate (19), respectively.

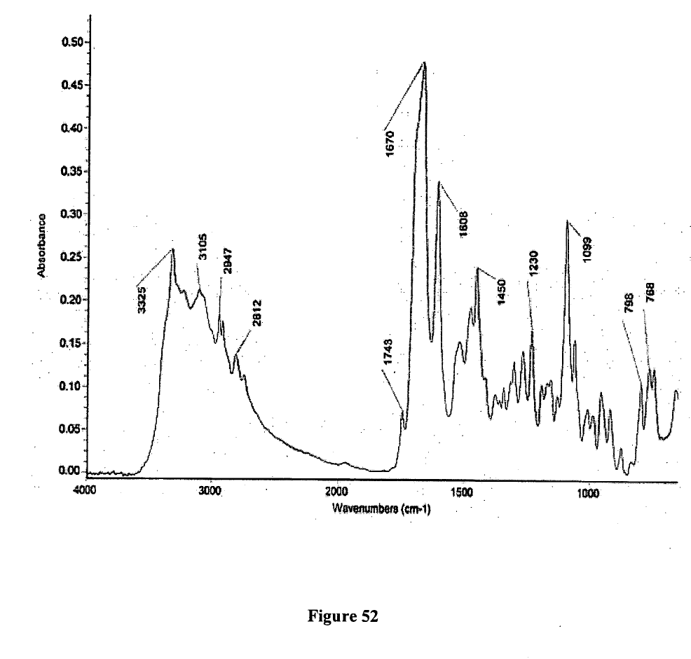

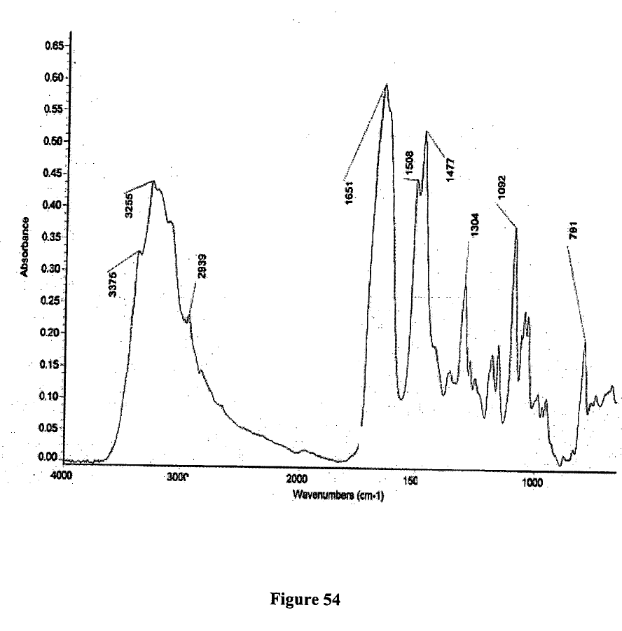

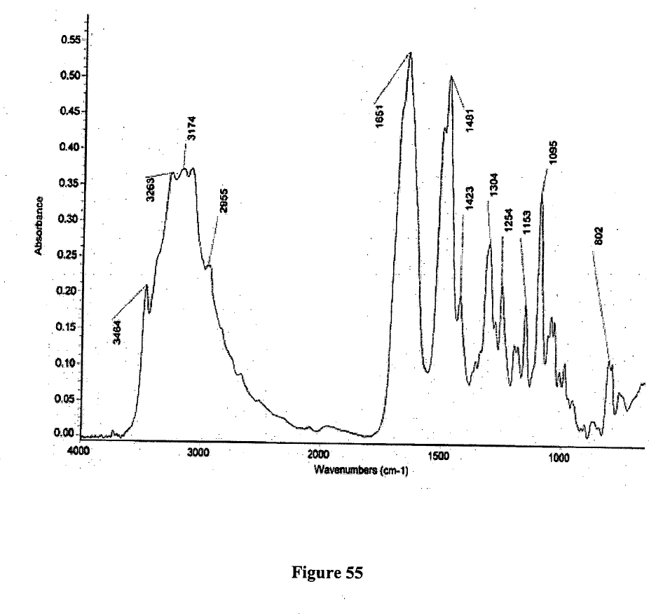

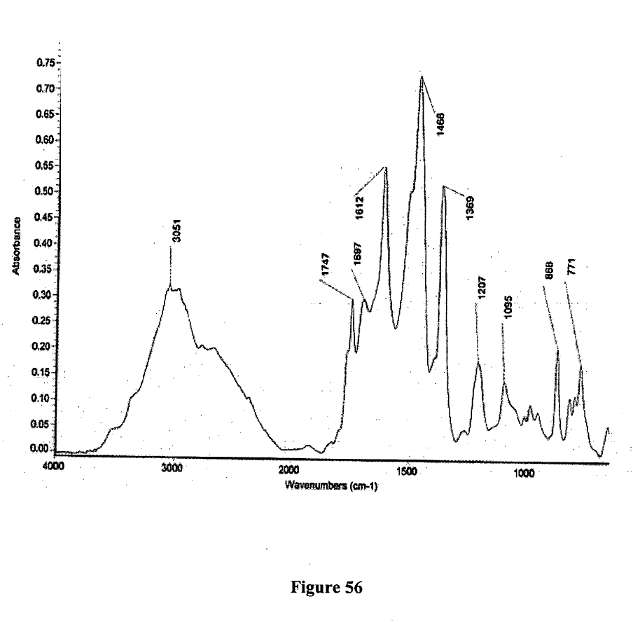

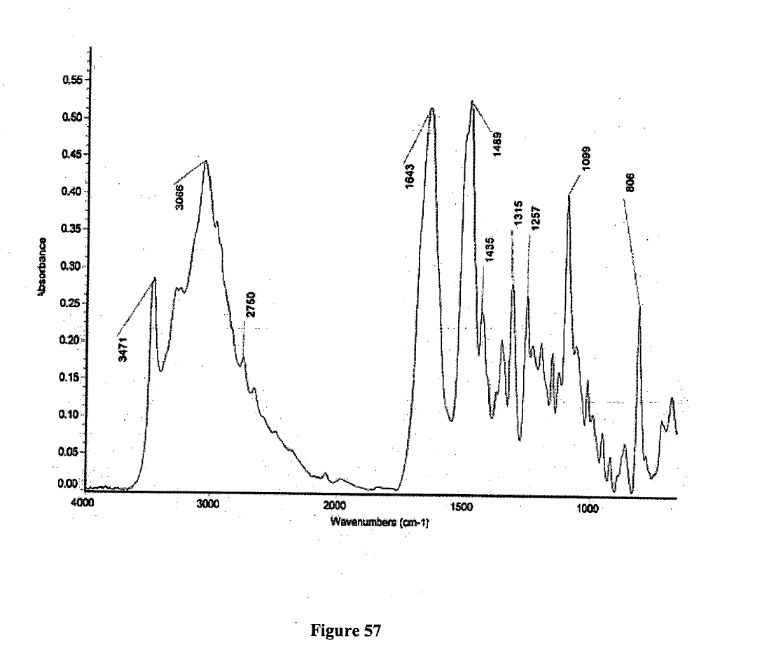

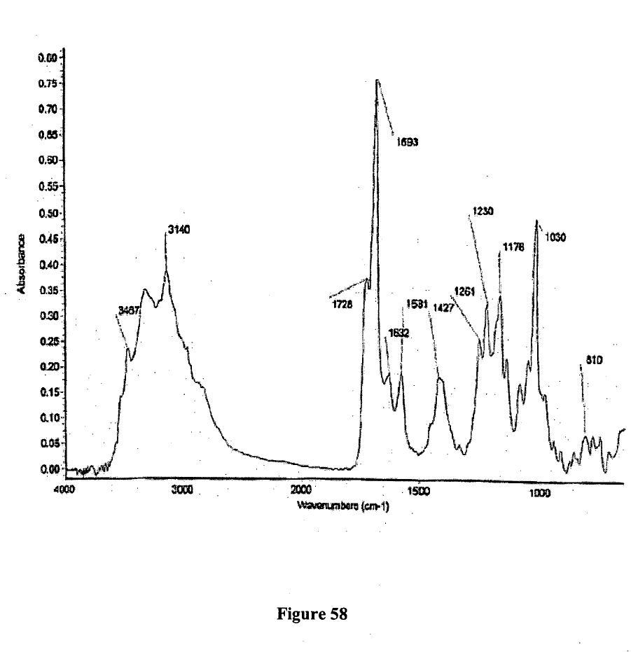

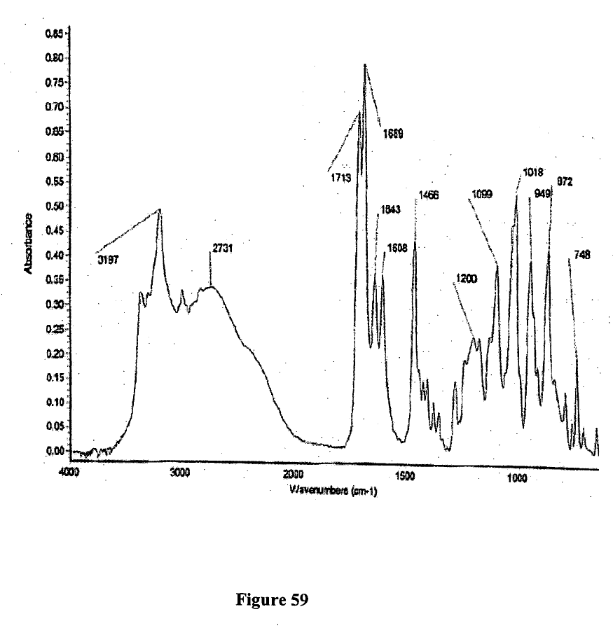

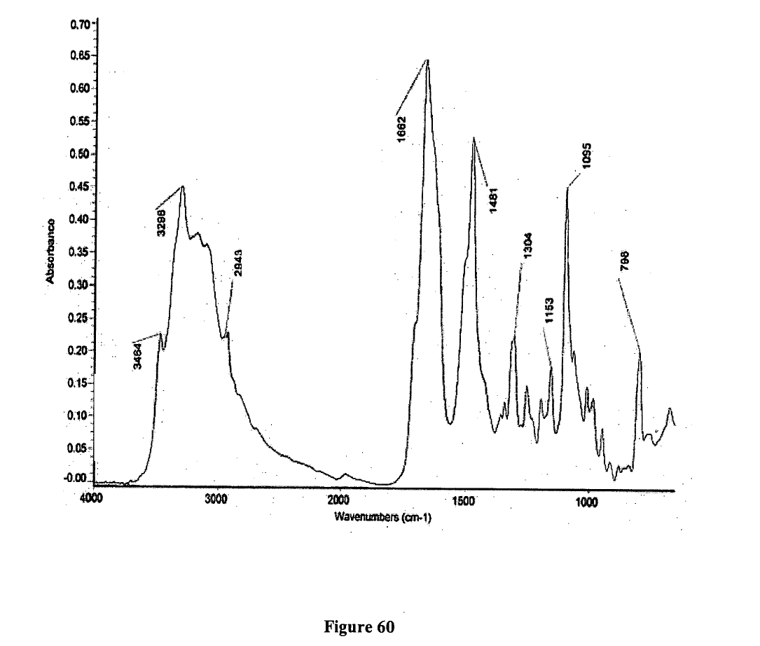

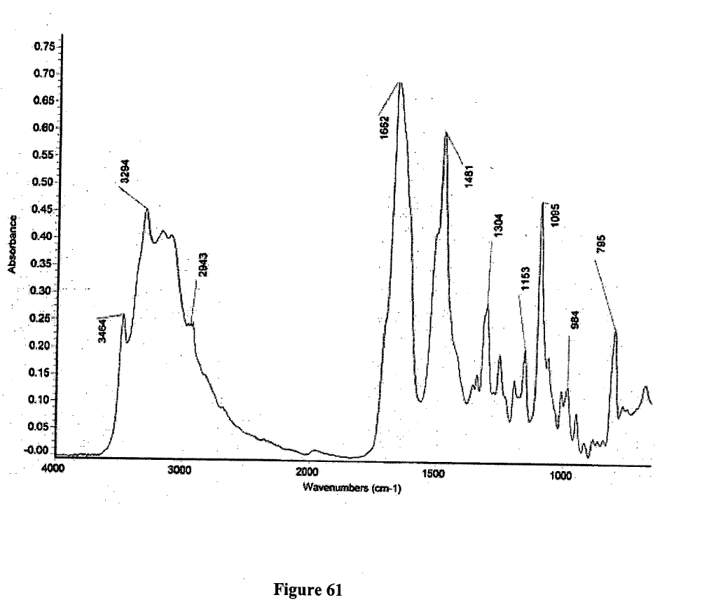

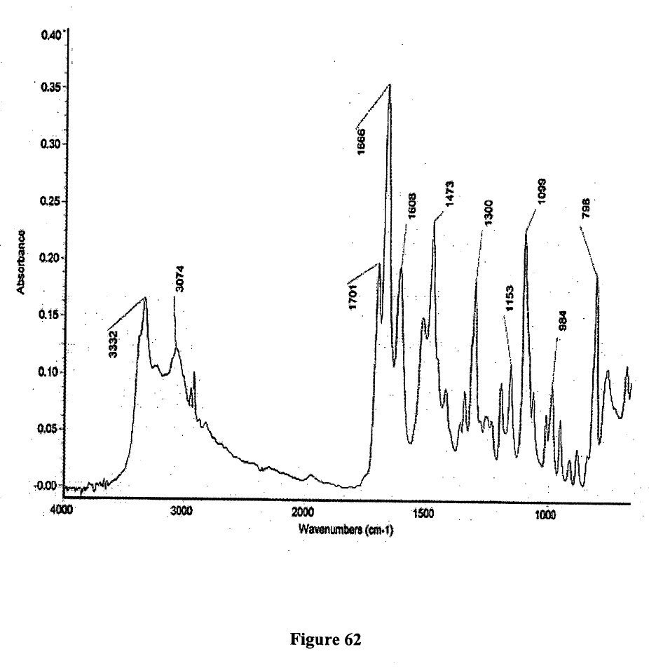

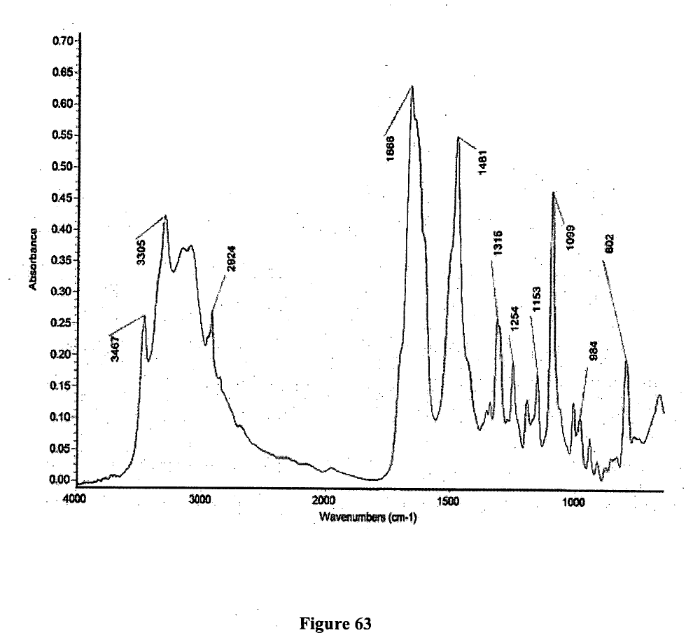

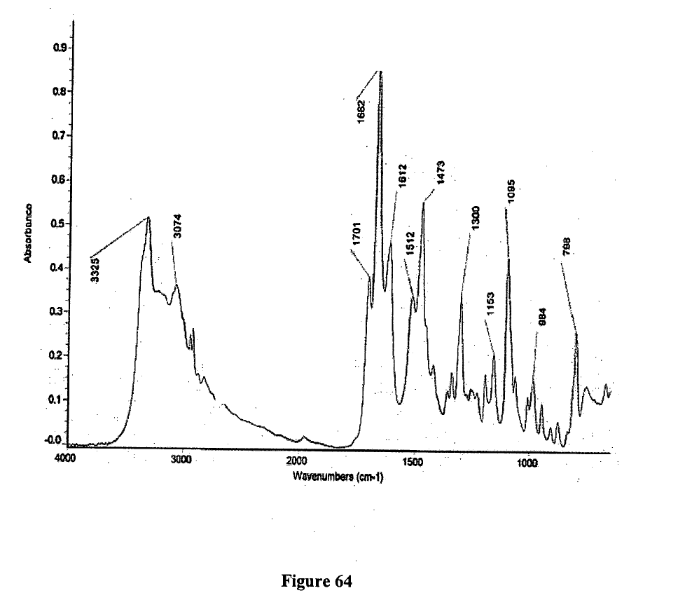

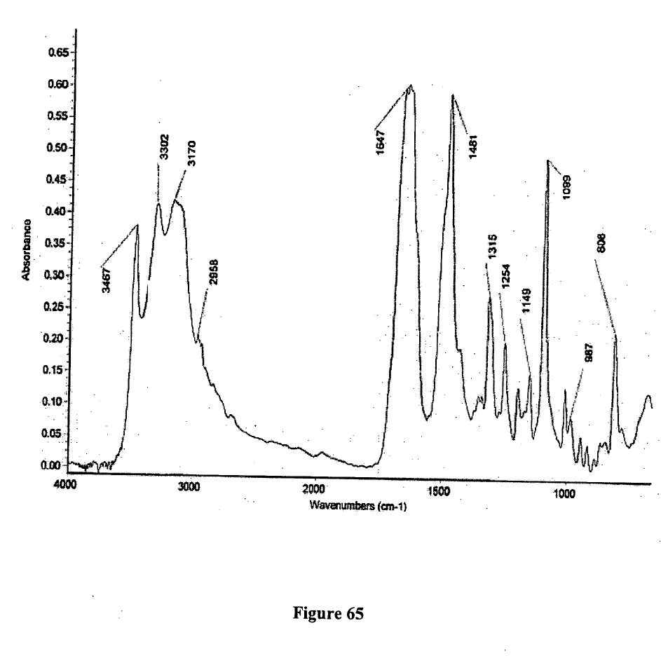

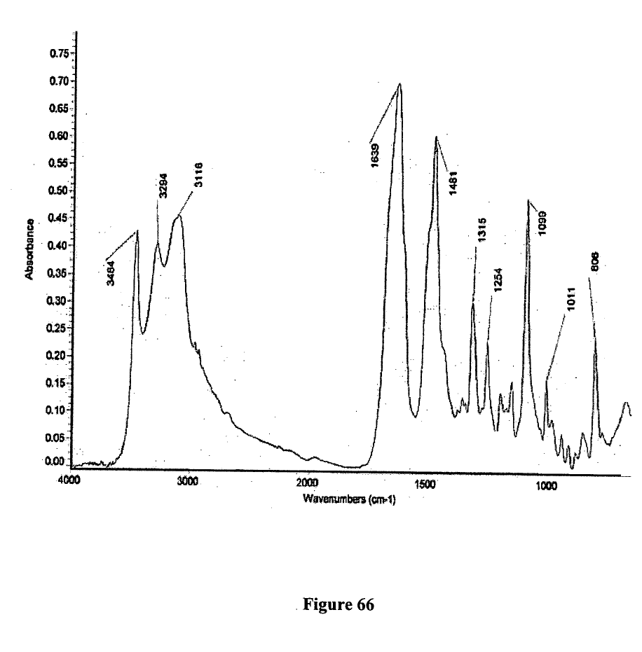

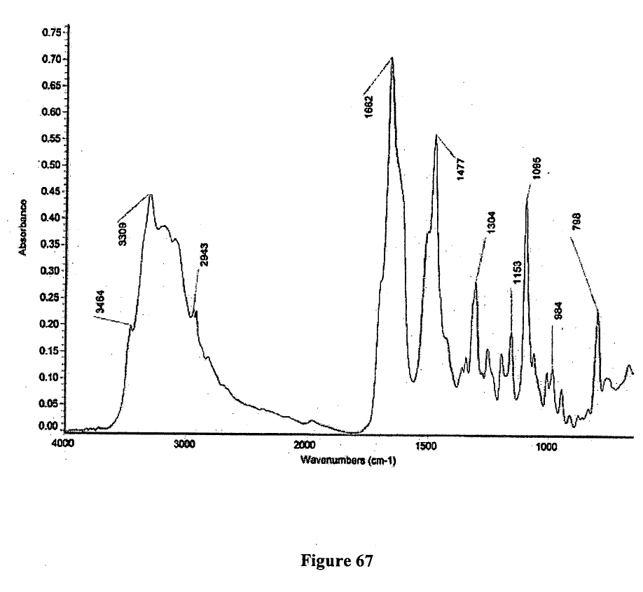

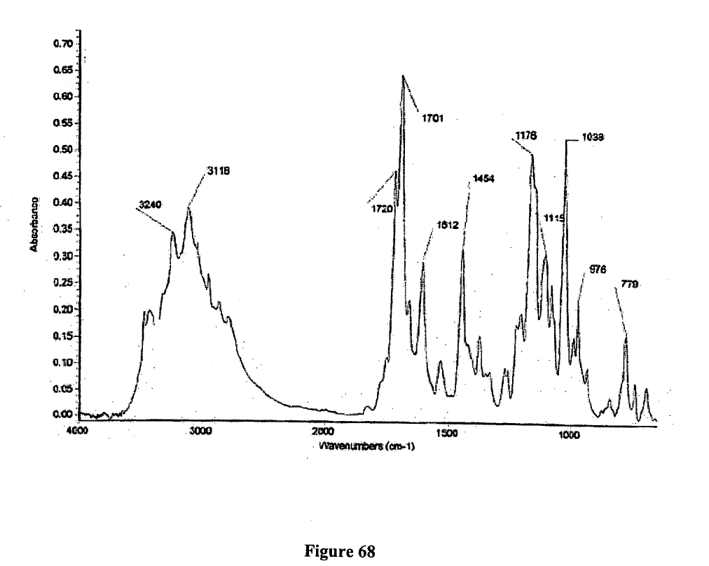

IR absorbance spectra also were obtained for certain embodiments of the instant invention. FIGS. 52-68 illustrate IR absorbance spectra for decitabine hydrochloride (3), decitabine mesylate (4), decitabine EDTA (5), decitabine L-aspartate (6), decitabine maleate (7), decitabine L-glutamate (8), decitabine sulfite (9), decitabine phosphate (10), decitabine tartrate (11), decitabine citrate (12), decitabine L-(+)-lactate (13), decitabine succinate (14), decitabine acetate (15), decitabine hexanoate (16), decitabine butyrate (17), decitabine propionate (18), and azacitidine mesylate (19), respectively.

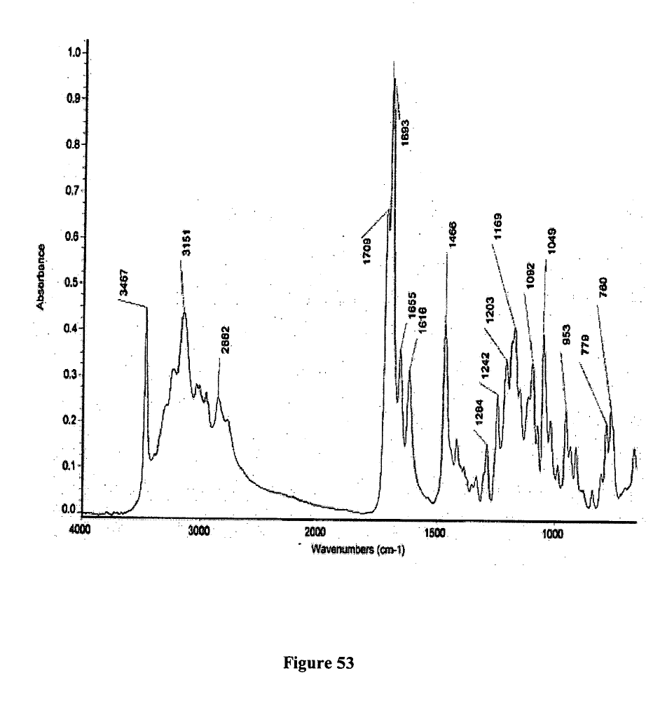

From the IR spectra for decitabine hydrochloride (3) (FIG. 52) and decitabine mesylate (4) (FIG. 53), one of skill in the art can see that all functional groups that exist in decitabine remain intact in decitabine hydrochloride and decitabine mesylate salts. A characteristically strong absorption for S═O (stretching vibration) appears at 1169 cm⁻¹ for decitabine mesylate (4) that does not exist for decitabine free base.

5. Summary of Analytical Data

Table 5 provides a summary of analytical data for certain embodiments relating to decitabine and azacitidine salts of the instant invention, including DSC, TGA, XRD and IR spectra for decitabine hydrochloride (3), decitabine mesylate (4), decitabine EDTA (5), decitabine L-aspartate (6), decitabine maleate (7), decitabine L-glutamate (8), decitabine sulfite (9), decitabine phosphate (10), decitabine tartrate (11), decitabine citrate (12), decitabine L-(+)-lactate (13), decitabine succinate (14), decitabine acetate (15), decitabine hexanoate (16), decitabine butyrate (17), decitabine propionate (18), and azacitidine mesylate (19), along with the corresponding Figures (discussed above). For comparison, decitabine free base (1), decitabine hydrate ('1), and azacitidine free base (2) data are also provided.

TABLE 5
Summary of analytical data for certain decitabine and azacitidine salts

| | | Melting Point | | | XRD | Distinctive |
|---|---|---|---|---|---|---|
| | | (° C.) | DSC | | Maxima^{c} | Absorption |
| # | Sample | (Decompose) | Endotherm^{a} | TGA^{b} Wt. Loss | (CPS @ θ-2θ°) | (cm⁻¹) |
| 1 | Decitabine | 190 | 203° C. | 0.032% @ 150° C. | — | — |
| | free base | | | | | |
| 1′ | Decitabine | — | 86.0° C., | 7.2% @ 150° C., | — | — |
| | Hydrate | | 94.9° C., | | | |
| | | | 198.4° C. | | | |
| 2 | Azacitidine | 230 | — | — | — | — |
| | free base | | | | | |
| 3 | Decitabine | 130 | 125 to 155° C. | 38.85% @ 160° C.; | 14.79°; 23.63°; | — |
| | HCl | | | 8.03% @ 200° C.; | 29.81° | |
| | | | | 3.95% @ 260° C.; | | |
| 4 | Decitabine | 125 | 125 to 140° C. | 15.29% @ 150° C.; | 8.52°; 22.09°; | 1169 (S═O) |
| | Mesylate | | | 14.06% @ 260° C. | 25.93° | |
| 5 | Decitabine | 230 | 50 to 90° C.; | 8.45% @ 200° C.; | 7.14°; 22.18°; | — |
| | EDTA | | 165 to 170° C.; | 39.14% @ 260° C. | 24.63° | |
| | | | 170 to 200° C. | | | |
| 6 | Decitabine L- | 190 | 30 to 100° C.; | 1.86% @ 80° C.; | 21.61°; 22.71°; | — |
| | Aspartate | | 170 to 195° C.; | 17.18% @ 220° C.; | 23.24° | |
| | | | 195 to 250° C. | 18.58% @ 260° C. | | |
| 7 | Decitabine | 210 | 95 to 130° C.; | 0.94% @ 80° C.; | 20.81°; 27.38°; | — |
| | Maleate | | 160 to 180° C. | 1.79% @ 100° C.; | 28.23° | |
| | | | | 32.66% @ 185° C.; | 28.23° | |
| | | | | 6.97% @ 100° C. | | |
| 8 | Decitabine L- | 180 | 50 to 100° C.; | 1.92% @ 80° C.; | 13.33°; 21.39°; | — |
| | Glutamate | | 175 to 195° C.; | 12.66% @ 200° C.; | 30.99° | |
| | | | 195 to 220° C. | 24.81% @ 260° C. | | |
| 9 | Decitabine | 220 | 100 to 140° C. | 26.31% @ 145° C.; | 15.73°; 19.23°; | 1176 (S═O) |
| | Sulfite | | | 31.98% @ 230° C.; | 22.67° | |
| | | | | 2.23% @ 260° C. | | |
| 10 | Decitabine | 118 | 130 to 145° C. | 22.36% @ 150° C.; | 17.09°; 21.99°; | — |
| | Phosphate | | | 19.18% @ 260° C. | 23.21° | |
| 11 | Decitabine | 202 | 60 to 110° C.; | 2.69% @ 90° C.; | 7.12°; 13.30°; | — |
| | (+)-L- | | 185 to 220° C. | 8.60% @ 200° C.; | 14.22° | |
| | Tartrate | | | 37.31% @ 260° C. | | |
| 12 | Decitabine | 202 | 30 to 100° C.; | 3.81% @ 80° C.; | 13.31°; 14.23°; | — |
| | Citrate | | 160 to 220° C. | 7.55% @ 200° C.; | 23.26° | |
| | | | | 39.02% @ 260° C. | | |
| 13 | Decitabine | 195 | 30 to 100° C.; | 3.08% @ 80° C.; | 13.27°; 21.13°; | — |
| | L-Lactate | | 160 to 210° C. | 8.93% @ 200° C.; | 23.72° | |
| | | | | 38.64% @ 260° C. | | |
| 14 | Decitabine | 210 | 50 to 100° C.; | 0.72% @ 185° C.; | 13.30°; 22.59°; | — |
| | Succinate | | 190 to 210° C. | 6.89% @ 205° C.; | 23.28° | |
| | | | | 35.02% @ 260° C. | | |
| 15 | Decitabine | 206 | 60 to 90° C., | 4.70% @ 75° C.; | 7.14°; 14.26°; | — |
| | Acetate | | 185 to 210° C. | 7.19% @ 195° C.; | 31.25° | |
| | | | | 39.17% @ 260° C. | | |
| 16 | Decitabine | 205 | 50 to 90° C., | 4.76% @ 75° C.; | 13.27°; 22.54°; | — |
| | Hexanoate | | 190 to 210° C. | 7.01% @ 195° C.; | 23.25° | |
| | | | | 37.92% @ 260° C. | | |
| 17 | Decitabine | 204 | 40 to 90° C., | 5.12% @ 75° C.; | 13.28°; 22.57°; | — |
| | Butyrate | | 190 to 210° C. | 6.87% @ 195° C.; | 23.27° | |
| | | | | 37.90% @ 260° C. | | |
| 18 | Decitabine | 200 | 50 to 110° C., | 4.74% @ 75° C.; | 13.29°; 22.52°; | — |
| | Propionate | | 190 to 210° C. | 7.35% @ 200° C.; | 23.27° | |
| | | | | 36.07% @ 260° C. | | |
| 19 | Azacitidine | 138 | 30 to 80° C.; | 2.44% @ 70° C.; | 18.58°; 23.03°; | 1169-1176 |
| | Mesylate | | 80 to 110° C.; | 5.56% @ 145° C.; | 27.97° | (S═O) |
| | | | 110 to 140° C. | 13.28% @ 220° C.; | | |
| | | | | 13.49% @ 260° C. | | |

^{a}Temperature maxima of endothermic events, ° C. (δH, J/g)
^{b}Weight changes are relative to the weight of the sample at the starting point of the specific weight change event
^{c}Three integrated intensity maxima (counts) are shown

6. Oral Administration of Decitabine Mesylate into Anemic Baboons

As described above, the present invention provides novel decitabine salts with improved chemical stability, solubility and bioavailability, especially for oral administration. In this example, we demonstrated that a decitabine salt, decitabine mesylate, orally administrated into anemic baboons (Papio anubis) is orally bioavailable and efficacious in increasing HbF and decreasing DNA methylation of the ε- and γ-globin genes in the animal models of sickle cell anemia.

It is known that increased levels of fetal hemoglobin (HbF) lessen the severity of sickle cell disease. Subcutaneous and intravenous administration of a drug that inhibits DNA methyltransferase, 5-aza-2-deoxycytidine (Decitabine), increased HbF levels in hydroxyurea-refractory sickle cell patients and experimentally-induced anemic baboons. It has been found that oral administration of the DNA methyltransferase inhibitor 5-azacytidine was only effective when combined with tetrahydrouridine, a cytidine deaminase inhibitor; in mice orally administered decitabine has only 9% bioavailability.

In this example, we demonstrated that decitabine mesylate could increase HbF and cause DNA demethylation when administered orally at doses 8-36 fold higher than effective doses given subcutaneously. Three baboons were rendered anemic by acute phlebotomy for ten days and maintained at an Hct of 20 during the course of drug treatment. HbF levels following the initial bleeding and prior to decitabine mesylate administration were 6.3-13.9%. Each baboon received a different orally administered dose of DAC mesylate (18.7 mg/kg/day; 9.35 mg/kg/day; 4.1 mg/kg/day) for ten days. Peak HbF levels achieved in animals receiving these three different doses were 67.8, 61.9, and 17.4, respectively. Peak HbF in the two animals receiving higher doses were comparable to levels observed in these animals following subcutaneous injection of a lower dose of decitabine (0.52 mg/kg/day). Bisulfite sequence analysis showed that methylation of the ε- and γ-globin genes was decreased>50% in animals treated with 18.7 mg/kg and 9.35 mg/kg doses, while minimal changes were observed in the animal treated with the lowest dose (4.1 mg/kg). Chromatin immunoprecipitation (ChIP) studies showed that the levels of acetylated histones H3 and H4 associated with the β-globin promoter were 5-6 fold higher than with the γ-globin promoter in bled animals. Following decitabine mesylate, equivalent levels of acetylated histones H3 and H4 were associated with the γ- and β-globin promoters in the two animals treated with the higher doses of drug. The results are summarized in Table 6 below. These studies thus demonstrate that orally administered decitabine mesylate increased HbF, reduced DNA methylation of the ε- and γ-globin genes, and increased acetylation of histones H3 and H4 associated with the γ-globin promoter in anemic baboons.

TABLE 6
Effect of Oral Decitabine Mesylate on HbF and DNA Methylation

| | | DAC Dose | ε-globin | γ-globin | HbF |
|---|---|---|---|---|---|
| Animal | Treatment | (mg/kg/day) | (% dmC) | (% dmC) | (%) |
| PA 6974 | Bled | 0 | 96.6 | 79.3 | 6.3 |
| | Oral Decitabine | 18.7 | 43.3 | 35.0 | 67.8 |
| | mesylate | | | | |
| PA 7002 | Bled | 0 | 86.6 | 71.7 | 13.9 |
| | Oral Decitabine | 9.35 | 46.6 | 34 | 61.9 |
| | mesylate | | | | |
| PA 7001 | Bled | 0 | 90.0 | 78.7 | 6.3 |
| | Oral Decitabine | 4.1 | 86.6 | 74.1 | 17.4 |
| | mesylate | | | | |

It can While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising a salt of decitabine in an oral dosage form.

2. The pharmaceutical composition of claim 1 wherein said salt is synthesized with an acid.

3. The pharmaceutical composition 2 wherein said acid has a pKₐ of about 5 or less.

4. The pharmaceutical composition of claim 2 wherein said acid has a pKₐ of about 4 or less.

5. The pharmaceutical composition of claim 2 wherein pKₐ of said acid ranges from about 3 to about −10.

6. The pharmaceutical composition of claim 2 wherein said acid is selected from the group consisting of hydrochloric, L-lactic, acetic, phosphoric, (+)-L-tartaric, citric, propionic, butyric, hexanoic, L-aspartic, L-glutamic, succinic, EDTA, maleic, and methanesulfonic acid.

7. The pharmaceutical composition of claim 2 wherein said acid is selected from the group consisting of HBr, HF, HI, nitric, nitrous, sulfuric, sulfurous, phosphorous, perchloric, chloric, and chlorous acid.

8. The pharmaceutical composition of claim 2 wherein said acid is a carboxylic acid or a sulfonic acid.

9. The pharmaceutical composition of claim 8 wherein said carboxylic acid is selected from the group consisting of ascorbic, carbonic, and fumaric acid.

10. The pharmaceutical composition of claim 8 wherein said sulfonic acid is selected from the group consisting of ethanesulfonic, 2-hydroxyethanesulfonic, and toluenesulfonic acid.

11. The pharmaceutical composition of claim 1 wherein said salt is a hydrochloride, mesylate, EDTA, sulfite, L-Aspartate, maleate, phosphate, L-Glutamate, (+)-L-Tartrate, citrate, L-Lactate, succinate, acetate, hexanoate, butyrate, or propionate salt.

12. The pharmaceutical composition of claim 1, wherein the oral dosage form is tablet, capsule, suspension or liquid.

13. The pharmaceutical composition of claim 1, wherein the pharmaceutical composition further comprises a pharmaceutically acceptable carrier.

14. The pharmaceutical composition of claim 13, wherein the pharmaceutically acceptable carrier is a binding agent selected from the group consisting of acacia gum, gelatin, polyvinylpyrrolidone, sorbitol, and tragacanth.

15. The pharmaceutical composition of claim 13, wherein the pharmaceutically acceptable carrier is a filler selected from the group consisting of calcium phosphate, glycine, lactose, maize-starch, sorbitol, and sucrose.

16. The pharmaceutical composition of claim 13, wherein the pharmaceutically acceptable carrier is a lubricant selected from the group consisting of magnesium stearate, polyethylene glycol, silica, and talc.

17. The pharmaceutical composition of claim 13, wherein the pharmaceutically acceptable carrier is selected from the group consisting of acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, and sorbic acid.

18. A kit, comprising:
a pharmaceutical composition according to claim 1.

19. The kit of claim 18, further comprising: a written instruction describing how to use the pharmaceutical composition.

20. A method of treating a disease associated with undesirable cell proliferation in a subject comprising:
orally administering to the subject in need thereof a pharmaceutically effective amount of a salt of claim 1.

21. The method of claim 20, wherein the salt is orally administered to the subject at a dose of 0.1-1000 mg/m² per day.

22. The method of claim 20, wherein the salt is orally administered to the subject at a dose of 1-200 mg/m² per day.

23. The method of claim 20, wherein the salt is orally administered to the subject at a dose of 1-100 mg/m² per day.

24. The method of claim 20, wherein the salt is orally administered to the subject at a dose of 1-50 mg/m² per day.

25. The method of claim 20, wherein the disease is selected from the group consisting of benign tumors, cancer, hematological disorders, atherosclerosis, insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

26. The method according to claim 25, wherein the cancer is selected from group consisting of breast cancer, skin cancer, bone cancer, prostate cancer, liver cancer, lung cancer, non-small cell lung cancer, brain cancer, cancer of the larynx, gall bladder, pancreas, rectum, parathyroid, thyroid, adrenal, neural tissue, head and neck, colon, stomach, bronchi, and kidney cancer, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic sarcoma, malignant hypercalcemia, renal cell tumor, polycythermia vera, adenocarcinoma, glioblastoma multiforma, leukemias, lymphomas, malignant melanomas, and epidermoid carcinomas.

27. The method of claim 20, wherein the disease is selected from the group consisting of myelodysplastic syndrome, leukemia, malignant tumors, and sickle-cell anemia.
